(19) United States
(12) Patent Application Publication
(10) Pub. No.: US 2024/0036045 A1
(43) Pub. Date: Feb. 1, 2024

(54) BLOOD-BASED PROTEIN BIOMARKER PANEL FOR EARLY AND ACCURATE DETECTION OF CANCER

(71) Applicants: Evan Laurence Busch, Somerville, MA (US); The Brigham and Women`s Hospital, Inc., Boston, MA (US)

(72) Inventors: Limor Cohen, Boston, MA (US); David R. Walt, Boston, MA (US); Evan Laurence Busch, Somerville, MA (US)

(21) Appl. No.: 18/268,034

(22) PCT Filed: Dec. 22, 2021
(86) PCT No.: PCT/US2021/064910

§ 371 Date: Jun. 16, 2023

Related U.S. Application Data
(60) Provisional application No. 63/129,432, filed on Dec. 22, 2020.

(51) Int. Cl. G01N 33/574

(52) U.S. Cl. CPC G01N 33/57415; G01N 2800/56; G01N 2800/50

(57) ABSTRACT

Methods and compositions for accurate blood biomarker panel-based detection of cancer, e.g., breast cancer, and sub-typing, e.g., using ultrasensitive immunoassays, e.g., digital ELISA.

### CLAIM OF PRIORITY

This application claims the benefit of U.S. Provisional Patent Application Ser. No. 63/129,432, filed on Dec. 22, 2020. The entire contents of the foregoing are hereby incorporated by reference.

### FEDERALLY SPONSORED RESEARCH OR DEVELOPMENT

This invention was made with Government support under Grant No. W81XWH-11-1-0814 awarded by the Department of Defense. The Government has certain rights in the invention.

### TECHNICAL FIELD

Described herein are methods and compositions for accurate blood biomarker panel-based detection of cancer, e.g., breast cancer, and subtyping, e.g., using ultrasensitive immunoassays, e.g., digital ELISA.

### BACKGROUND

Breast cancer is the second leading cause of cancer death in females in the United States (1).

### SUMMARY

Described herein are methods and compositions for accurate blood biomarker panel-based detection of cancer, e.g., breast cancer, and subtyping, e.g., using ultrasensitive immunoassays, e.g., digital ELISA, on blood samples. Thus provided herein are methods that include obtaining a sample comprising blood (e.g., whole blood, serum, or plasma) from a subject, and determining a level of at least 2, 3, 4, 5, 10, 15, 20, or all 24 biomarkers as listed in Table Ain the sample. In some embodiments, the biomarkers comprise at least MICA, CA125, and CD25. In some embodiments, the biomarkers comprise at least HER3, HSP70, CYR61, and LCN2. In some embodiments, the biomarkers comprise at least ER, HER3, HER4, CXCL10, CYR61, P21, MICA, CD25, IL-6, and CA125.

In some embodiments, the methods include calculating a score for the subject based on the level of the biomarkers, wherein a score above a threshold score indicates that the subject has or is at risk of developing cancer.

In some embodiments, the methods include calculating a score for the subject based on the level of the biomarkers, and comparing the score to subtype reference scores for known subtypes of breast cancer and identifying a subject who has a score that is comparable to the subtype reference as having that subtype of breast cancer.

In some embodiments, the methods include recommending or sending the subject for additional evaluation, e.g., by imaging and/or biopsy.

In some embodiments, the methods include administering a treatment for breast cancer to a subject who has been identified as having or at risk of developing breast cancer. In some embodiments, the treatment comprises chemotherapy, hormone therapy, immunotherapy, radiation, or surgical resection.

In some embodiments, determining a level of biomarkers comprises using digital ELISA, e.g., Single-Molecule Arrays (SIMOA); Meso Scale Discovery (MSD); Single-Molecule Counting (SMC); LUMINEX; SOMAscan Assays; mass spectrometry (e.g., MALDI-MS), and/or mass cytometry (e.g., CyTOF).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-D: Selection and initial validation of the biomarker panel in tumor tissue and blood. A. List of biomarkers. B. PCA of mRNA expression for the 24 biomarkers measured in various human tumors (9,860 cancer subjects of which 1,084 are breast cancer subjects) from the TCGA database. Samples were assessed by RNA-seq. C. Histogram of principal component 1 (data from B). D. PCA of protein levels for 24 biomarkers measured in serum from healthy (n=24) and breast cancer subjects (n=25). Serum samples were measured using Simoa assays.

FIGS. 2A-D: Distinguishing between healthy and breast cancer subjects using blood biomarkers. A. ROC curves for a model using a panel of 24 biomarkers plus age, and a model using age alone. B. ROC curve for a model using a panel of four biomarkers plus age. The four biomarkers are HER3, HSP70, CYR61, and LCN2. C. ROC curve for HSP70 plus age. For panels A-C, the 95% confidence intervals are shown in parentheses. D. Decision curves based on 1) the panel of 24 biomarkers plus age, 2) the panel of four biomarkers plus age, 3) HSP70 plus age, 4) age alone, 5) classifying all patients as cancer (treat all), and 6) classifying no patients as cancer (treat none).

FIGS. 3A-E: Subtype analysis using the candidate biomarker. A. Model performance for accurately classifying different breast cancer subtypes as cancer. B. ROC curves for healthy and ER+breast cancer subjects and healthy and TNBC subjects using the panel of 24 biomarkers plus age and the panel of four biomarkers plus age. C. PCA of mRNA expression levels in breast cancer tumors using our biomarker candidates. Luminal A (n=412), Luminal B (n=174), Normal (n=25), TNBC (n=136), HER2 (n=65). D. Percent contribution of each marker to the PCA shown in C. E. ROC curves for the protein panel in blood using the ten most informative markers (ER, HER3, HER4, CXCL10, CYR61, P21, MICA, CD25, IL-6, and CA125) and the top three most informative markers (MICA, CA125, and CD25). Hormone positive (n=81) and TNBC (n=10). For panels B and E, the 95% confidence intervals are shown in parentheses.

FIGS. 4A-D. Digital ELISA based on arrays of femtoliter-sized wells. 25 (A, B) Single protein molecules are captured and labeled on beads using standard ELISA reagents (A), and beads are loaded into femtoliter-volume well arrays (B). (C) SEM of a section of a femtoliter-volume well array after bead loading. (D) Fluorescence image of a section of the femtoliter-volume well array after signals from single enzymes are generated. Only a fraction of beads possess enzyme activity, indicating a single, bound protein molecule.

FIG. 5: Simoa assay calibration curves and detection limits

FIG. 6: Simoa assay dilution linearity

FIG. 7: Simoa assay spike and recovery

FIG. 8: Biomarker levels in cancer and healthy subjects

FIG. 9: Calibration plots for prediction models

FIG. 10: XY scatterplots of informative markers

FIG. 11: Correlation between biomarker levels and age in healthy subjects

FIG. 12: Variable importance for the model used to distinguish between different subtypes in blood.

### DETAILED DESCRIPTION

Large-scale breast cancer screening programs have been widely implemented because early detection and treatment can improve patient outcomes (2). However, detecting breast cancer early and accurately is challenging due to limitations in conventional detection methods, such as mammography, which suffer from high false-positive and false-negative rates (3-11). Additionally, current screening methods do not provide any disease-relevant molecular information and thus are limited in their ability to distinguish between benign and malignant breast tumors. Since breast cancer is a highly heterogeneous disease, detection methods that provide molecular information are promising for early and accurate detection. Thus, advances in breast cancer detection can reduce patient morbidity by preventing unnecessary invasive biopsies, which arise from screen-detected false positives. Advances in detection methods will also enable timely intervention for cancers that require treatment, thereby improving patient outcomes.

Liquid biopsies for cancer detection are particularly promising since they provide molecular information and are minimally invasive (12, 13). Currently, efforts to develop liquid biopsies for breast cancer mainly rely on detecting circulating tumor DNA (ctDNA) and circulating tumor cells (CTCs) (14-16). However, applying these two classes of biomarkers to early cancer detection is challenging because the tumor must be relatively large to produce sufficient quantities of ctDNA or CTCs that can be detectable in blood (17-19). Proteins are particularly promising biomarkers since they are directly involved in biological processes that are dysregulated in disease and are also abundant in the cell. Furthermore, plasma proteins have been shown to be indicators of health status (20, 21). Previous studies have developed blood tests for breast cancer detection; however, these attempts have limited accuracy, particularly for early stage breast cancer detection (22, 23). Thus, developing a test using circulating proteins may improve our ability to accurately detect breast cancer (24).

Described herein is a blood protein biomarker panel for breast cancer detection. In some embodiments, the methods use analytically robust Single Molecule Array (Simoa) immunoassays (25, 26). Using gene expression data from The Cancer Genome Atlas (TCGA) (27), we showed that the biomarkers were able to distinguish between breast cancer and other types of cancer in tumor tissues. We then developed and analytically validated assays for these biomarkers in blood and showed that the panel can distinguish between healthy and breast cancer patients in a small preliminary cohort (n=49). We then applied the biomarker panel to a second, larger cohort of healthy and newly diagnosed, treatment-naïve breast cancer patients (n=197).

The results reported here provide evidence that circulating proteins can accurately detect breast cancer. This was especially encouraging given that most of the breast cancer subjects had tumors consistent with early-stage disease, an important consideration for detection and screening methods. For the model using 24 biomarkers plus age, the overall AUC was 0.95 (95% CI 0.92-0.98) and 88% of subjects were correctly classified, with 87% sensitivity and 90% specificity. This compares favorably with mammography, which has a false-negative rate of about 20% (7-11). Additionally, over 50% of patients screened annually for 10 years in the U.S. will have a false-positive mammogram, which requires further evaluation with a biopsy (3-6). Decreasing the mammogram screen-detected false-positives would reduce unnecessary invasive diagnostic surgical procedures and overall patient morbidity. Furthermore, the model using the 24 biomarkers plus age showed greater net benefit across a wide range of threshold probabilities compared to the other models, suggesting that diagnostic decisions made with information from the panel of markers could be superior to those made without it.

We also downselected the most informative markers and showed that HER3, HSP70, CYR61, and LCN2 are especially important biomarkers, with an AUC of 0.87 (95% CI 0.81-0.92) for this four biomarker panel.

The panel of protein biomarkers substantially outperformed any single protein. We observed an AUC of 0.95 for a panel using the 24 biomarkers plus age, 0.87 for a panel using the four most informative markers plus age, and 0.77 for HSP70 and age, which was the best-performing single marker. The full panel had better discrimination, calibration, and improvement in diagnostic decision-making by net benefit (51) than the four biomarker panel using the most informative markers. Furthermore, the panels performed substantially better than any individual marker. For a given biomarker, the concentrations in the breast cancer and healthy groups largely overlapped, indicating that the ability to distinguish between breast cancer and healthy subjects depends on the cumulative effect of multiple markers. These results indicate that the full panel is critical for accurately detecting breast cancer.

Finally, as shown herein some of the biomarkers can be used for distinguishing between molecular subtypes of breast cancer. MICA, CA125, and

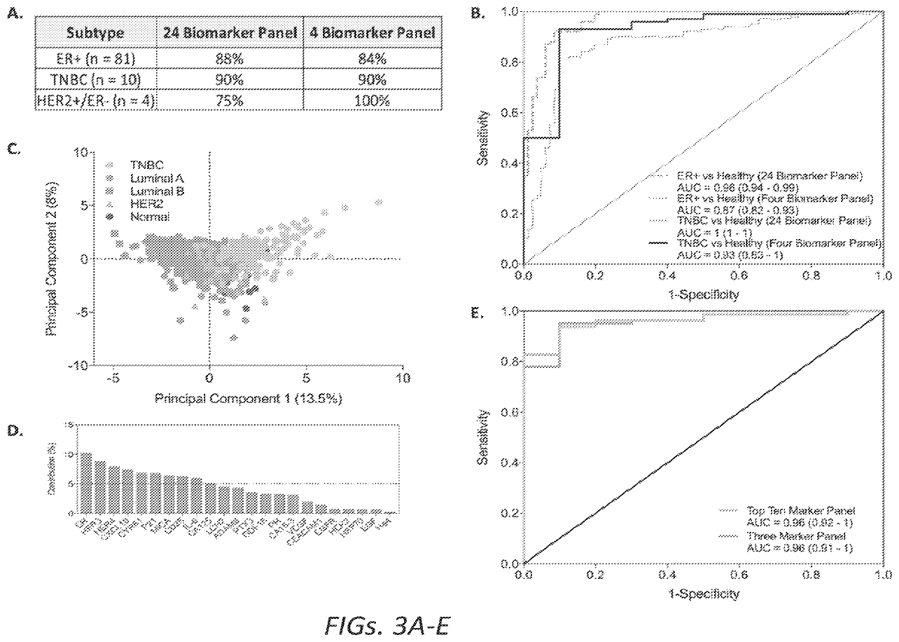

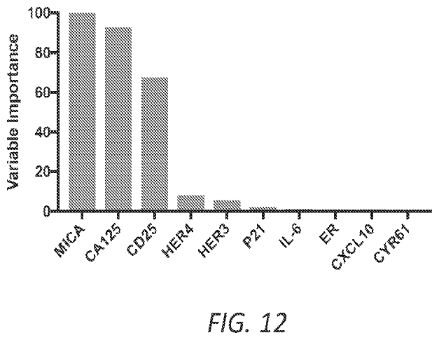

CD25 were the top three most informative protein biomarkers in blood for subtyping (FIG. 12), with an AUC of 0.96 (95% CI 0.91-1.00) using this three-marker panel (FIG. 3E).

The blood tests described herein can be used, e.g., individually or in combination with another clinical modality, such as mammography, to improve the accuracy of breast cancer screening.

### Methods of Diagnosis

Included herein are methods for diagnosing breast cancer, and/or determining the subtype of breast cancer present in a subject. The methods rely on detection of a biological marker or a plurality of protein biological markers as described herein, e.g., as shown in Table A. In some embodiments, the present methods provide blood tests for breast cancer detection and diagnosis using circulating protein biomarkers.

Proteins are responsible for cell growth, proliferation, signaling, motility, metabolic processes, and regulate tumorigenesis via cell adhesion, invasion, and migration. Additionally, proteins modulate the immune system's response to cancer.

Therefore, protein signatures involved in breast cancer pathophysiology are extremely promising for breast cancer detection and diagnosis. Provided herein is a panel of protein biomarkers associated with breast cancer. These biomarkers are involved in various biological processes including angiogenesis, proliferative signaling, and metastasis.

TABLE A
Breast Cancer Biomarkers

| Protein | Full name | RefSeq ID - Human | Description |
|---|---|---|---|
| ADAM8 | ADAM | NP_001100.3 (isoform 1 | Promotes breast cancer |
| | metallopeptidase | precursor) | development and brain |
| | domain 8 (or | NP_001157961.1 (isoform 2 | metastasis.⁵⁹ |
| | disintegrin and | precursor) | |
| | metalloproteinase | NP_001157962.1 (isoform 3 | |
| | domain-containing | precursor) | |
| | protein 8) | | |
| CA125 | Cancer antigen 125 | NP_078966.2 | Associated with breast cancer |
| | or mucin-16 | | metastasis^{62, 63} |
| CA15-3 | Cancer antigen 15-3 | NP_002447.4 (isoform 1 | Overexpressed in cancer cells |
| | or mucin-1 | precursor), NP_001018016.1 | and shed into the blood. |
| | | (isoform 2 precursor), | Elevated in metastatic breast |
| | | NP_001018017.1 (isoform 3 | cancer and is currently in |
| | | precursor), NP_001037855.1 | clinical use to monitor |
| | | (isoform 5 precursor), | response to treatment and |
| | | NP_001037856.1 (isoform 6 | recurrence.⁶⁷ |
| | | precursor), NP_001037857.1 | |
| | | (isoform 7 precursor), | |
| | | NP_001037858.1 (isoform 8 | |
| | | precursor), NP_001191214.1 | |
| | | (isoform 9 precursor), | |
| | | NP_001191215.1 (isoform 10 | |
| | | precursor), NP_001191216.1 | |
| | | (isoform 11 precursor), | |
| | | NP_001191217.1 (isoform 12 | |
| | | precursor), NP_001191218.1 | |
| | | (isoform 13 precursor), | |
| | | NP_001191219.1 (isoform 14 | |
| | | precursor), NP_001191220.1 | |
| | | (isoform 15 precursor), | |
| | | NP_001191221.1 (isoform 16 | |
| | | precursor), NP_001191222.1 | |
| | | (isoform 17 precursor), | |
| | | NP_001191223.1 (isoform 18 | |
| | | precursor), NP_001191224.1 | |
| | | (isoform 19 precursor), | |
| | | NP_001191225.1 (isoform 20 | |
| | | precursor), NP_001358649.1 | |
| | | (isoform 22 precursor) | |
| CA19-9 | Carbohydrate | N/A | Present on the surface of |
| | antigen 19-9 | | some cancer cells and can be |
| | | | shed into the blood. |
| | | | Commonly used as a tumor |
| | | | marker for various types of |
| | | | cancer.⁷¹ |
| CD25 | Interleukin-2 | NP_000408.1 (isoform 1 | Immune marker associates |
| | receptor alpha | precursor), NP_001295171.1 | with breast cancer.⁷⁴ |
| | chain (also called | (isoform 2 precursor), | |
| | CD25) | NP_001295172.1 (isoform 3 | |
| | | precursor) | |
| CEACA | carcinoembryonic | NP_001703.2 (isoform 1 | Cell adhesion molecule |
| M1 | antigen-related cell | precursor), NP_001020083.1 | associated with breast cancer |
| | adhesion molecule | (isoform 2 precursor), | metastasis.⁷⁶ |
| | 1 | NP_001171744.1 (isoform 3 | |
| | | precursor), NP_001171742.1 | |
| | | (isoform 4 precursor), | |
| | | NP_001171745.1 (isoform 5 | |
| | | precursor), NP_001192273.1 | |
| | | (isoform 6 precursor) | |
| CXCL10 | C-X-C motif | NP_001556.2 | Immune marker associates |
| | chemokine ligand | | with breast cancer.⁷⁹ |
| | 10 | | |
| CYR61 | cysteine rich | NP_001545.2 | Involved in cellular growth |
| | angiogenic inducer | | and differentiation. Has been |
| | 61 (also known as | | shown to play an important |
| | CCN family member | | role in breast cancer |
| | 1 precursor) | | progression.^{81, 82} |
| EGF | Epidermal growth | NP_001954.2 (isoform 1 | Regulates epithelial- |
| | factor | precursor), NP_001171601.1 | mesenchymal transition, |
| | | (isoform 2 precursor), | migration, and tumor invasion |
| | | NP_001171602.1 (isoform 3 | in breast cancer.⁸⁴ |
| | | precursor), NP_001343950.1 | |
| | | (isoform 4 precursor) | |
| EGFR | Epidermal growth | NP_005219.2 (isoform a | Epidermal growth factor |
| | factor receptor | precursor), NP_958439.1 | receptor (EGFR) plays a role in |
| | | (isoform b precursor), | tumor progression and |
| | | NP_958440.1 (isoform c | resistance to therapy.⁸⁶ |
| | | precursor), NP_958441.1 | |
| | | (isoform d precursor), | |
| | | NP_001333826.1 (isoform e | |
| | | precursor), NP_001333827.1 | |
| | | (isoform f precursor), | |
| | | NP_001333828.1 (isoform g | |
| | | precursor), NP_001333829.1 | |
| | | (isoform h precursor), | |
| | | NP_001333870.1 (isoform i | |
| | | precursor) | |
| ER | Estrogen receptor | NP_000116.2 (isoform 1), | Estrogen and progesterone |
| | | NP_001278159.1 (isoform 2), | receptors cause cancer cells |
| | | NP_001278170.1 (isoform 3), | grow in response to the |
| | | NP_001315029.1 (isoform 4), | hormone estrogen and |
| | | NP_001372499.1 (isoform 5), | progesterone, respectively. |
| | | (isoform 6), (isoform 7) | The majority of breast |
| PR | Progesterone | NP_001189403.1 (isoform A), | cancers are ER/PR positive. ER |
| | receptor | NP_000917.3 (isoform B), | is also a target for endocrine |
| | | NP_001258090.1 (isoform C), | therapy.⁸⁸ |
| | | NP_001258091.1 (isoform D) | |
| GDF15 | Growth | NP_004855.2 | Growth differentiation factor |
| | differentiation | | that mediates epithelial- |
| | factor 15 | | mesenchymal transition and |
| | | | breast cancer invasion.^{60, 61} |
| He4 | Human Epithelial | NP_006094.3 | Associated with breast |
| | Protein 4 (HE4) (or | | carcinogenesis or tumor |
| | WAP four-disulfide | | progression.⁶⁴⁻⁶⁶ |
| | core domain | | |
| | protein 2 precursor) | | |
| HER2 | erb-b2 receptor | NP_004439.2 (isoform a | ERBB family receptor tyrosine |
| | tyrosine kinase 2 | precursor), NP_001005862.1 | kinases are overexpressed in |
| | | (isoform b precursor), | a substantial number of |
| | | NP_001276865.1 (isoform c | breast cancers, commonly in |
| | | precursor), NP_001276866.1 | patients with lymph node |
| | | (isoform d precursor), | metastasis.⁶⁸⁻⁷⁰ |
| | | NP_001276867.1 (isoform e | |
| | | precursor), NP_001369713.1 | |
| | | (isoform f precursor), | |
| | | NP_001369714.1 (isoform g | |
| | | precursor), NP_001369715.1 | |
| | | (isoform h precursor), | |
| | | NP_001369716.1 (isoform i | |
| | | precursor), NP_001369717.1 | |
| | | (isoform j precursor), | |
| | | NP_001369718.1 (isoform k | |
| | | precursor), NP_001369719.1 | |
| | | (isoform l precursor), | |
| | | NP_001369720.1 (isoform m | |
| | | precursor), NP_001369721.1 | |
| | | (isoform n precursor), | |
| | | NP_001369722.1 (isoform o | |
| | | precursor), NP_001369723.1 | |
| | | (isoform p precursor), | |
| | | NP_001369724.1 (isoform q | |
| | | precursor), NP_001369725.1 | |
| | | (isoform r precursor), | |
| | | NP_001369726.1 (isoform s | |
| | | precursor), NP_001369727.1 | |
| | | (isoform t precursor), | |
| | | NP_001369728.1 (isoform u | |
| | | precursor), NP_001369729.1 | |
| | | (isoform v precursor), | |
| | | NP_001369730.1 (isoform w | |
| | | precursor), NP_001369731.1 | |
| | | (isoform x precursor), | |
| | | NP_001369732.1 (isoform y | |
| | | precursor), NP_001369733.1 | |
| | | (isoform z precursor), | |
| | | NP_001369734.1 (isoform aa | |
| | | precursor), NP_001369735.1 | |
| | | (isoform bb precursor) | |
| HER3 | erb-b2 receptor | NP_001973.2 (isoform 1 | |
| | tyrosine kinase 3 | precursor), NP_001005915.1 | |
| | | (isoform s precursor) | |
| HER4 | erb-b2 receptor | NP_005226.1 (isoform JM- | |
| | tyrosine kinase 4 | a/CVT-1 precursor), | |
| | | NP_001036064.1 (isoform JM- | |
| | | a/CVT-2 precursor) | |
| HSP70 | heat shock protein | NP_005336.3 | Overexpressed in many |
| | family A (Hsp70) | | breast cancers and associated |
| | member 1A | | with poor prognosis.^{72, 73} |
| IL-6 | Interleukin 6 | NP_000591.1 (isoform 1 | Immune marker associates |
| | | precursor), NP_001305024.1 | with breast cancer.⁷⁵ |
| | | (isoform 2 precursor), | |
| | | NP_001358025.1 (isoform 3 | |
| | | precursor) | |
| LCN2 | Lipocalin 2 | NP_005555.2 | Promotes breast cancer |
| | | | progression and associated |
| | | | with invasive breast |
| | | | cancer.^{77, 78} |
| MICA | MHC class I | NP_000238.1 (isoform 1), | Immune marker associates |
| | polypeptide-related | NP_001170990.1 (isoform 2), | with breast cancer.⁸⁰ |
| | sequence A | NP_001276081.1 (isoform 3), | |
| | | NP_001276083.1 (isoform 4) | |
| P21 | Cyclin dependent | NP_000380.1 (isoform 1), | Loss of p21 expression is |
| | kinase inhibitor 1A | NP_001278478.1 (isoform 2), | associated with a high |
| | | NP_001361439.1 (isoform 3), | percentage of breast cancers |
| | | NP_001361440.1 (isoform 4), | and lack of response to |
| | | NP_001361441.1 (isoform 5) | certain hormone therapies.⁸³ |
| PTX3 | Pentraxin 3 | NP_002843.2 | Immune marker associates |
| | | | with breast cancer.⁸⁷ |
| VEGF | VEGF-A, VEGF-B, | NP_001020537.2 (isoform a), | Vascular endothelial growth |
| | VEGF-C, VEGF-D, | NP_003367.4 (isoform b), | factor (VEGF), an angiogenic |
| | VEGF-E, and PIGF | NP_001020538.2 (isoform c), | growth factor, is commonly |
| | | NP_001020539.2 (isoform d), | expressed in breast cancer |
| | | NP_001020540.2 (isoform e), | and promotes metastasis.⁸⁹ |
| | | NP_001020541.2 (isoform f), | |
| | | NP_001028928.1 (isoform g), | |
| | | NP_001165093.1 (isoform h), | |
| | | NP_001165094.1 (isoform l | |
| | | precursor), NP_001165095.1 | |
| | | (isoform j precursor), | |
| | | NP_001165096.1 (isoform k | |
| | | precursor), NP_001165097.1 | |
| | | (isoform VEGF-A precursor), | |
| | | NP_001165098.1 (isoform m | |
| | | precursor), NP_001165099.1 | |
| | | (isoform n precursor), | |
| | | NP_001165100.1 (isoform o | |
| | | precursor), NP_001165101.1 | |
| | | (isoform p precursor), | |
| | | NP_001191313.1 (isoform q | |
| | | precursor), NP_001191314.1 | |
| | | (isoform r), NP_001273973.1 | |
| | | (isoform s), NP_001303939.1 | |
| | | (isoform VEGF-Ax precursor) | |

In some embodiments, the methods include determining levels of at least 3, 4, 5, 10, 15, 20, or all 24 of the biomarkers in Table A. In some embodiments, the biomarkers comprise at least MICA, CA125, and CD25. In some embodiments, the biomarkers comprise at least HER3, HSP70, CYR61, and LCN2. In some embodiments, the biomarkers comprise at least ER, HER3, HER4, CXCL10, CYR61, P21, MICA, CD25, IL-6, and CA125. In some embodiments, where multiple isoforms of a biomarker exist, a method that detects all of the isoforms is used.

The methods include obtaining a sample from a subject, and evaluating the presence and/or level of a breast cancer biomarker in the sample.

The methods can also include comparing the presence and/or level with one or more references, e.g., a control reference that represents a normal level of the breast cancer biomarker, e.g., a level in an unaffected subject, and/or a disease reference that represents a level of the proteins associated with breast cancer, e.g., a level in a subject having breast cancer. In some embodiments, the level provides for differential diagnosis, e.g., is a level in a subject having a known type of breast cancer (e.g., ER+ or TNBC). Suitable reference values can include those shown in Table 1.

As used herein the term “sample”, when referring to the material to be tested for the presence of a biological marker using the method of the invention, includes inter alia whole blood, plasma, or serum. If needed, various methods are well known within the art for the identification and/or isolation and/or purification of a biological marker from a sample. An “isolated” or “purified” biological marker is substantially free of cellular material or other contaminants from the cell or tissue source from which the biological marker is derived, i.e. partially or completely altered or removed from the natural state through human intervention. For example, proteins contained in the sample can be isolated according to standard methods, for example using lytic enzymes, chemical solutions, or isolated by protein-binding resins following the manufacturer's instructions.

The presence and/or level of a protein can be evaluated using methods known in the art. In preferred embodiments, the methods include the use of highly sensitive or ultrasensitive and preferably multiplex detection methods including Meso Scale Discovery (MSD); Single-Molecule Arrays (SIMOA); Single-Molecule Counting (SMC); LUMINEX; SOMAscan Assays; mass spectrometry (e.g., MALDI-MS) and mass cytometry (e.g., CyTOF) (see, e.g., Cohen and Walt, Chem. Rev. 2019, 119, 293-321).

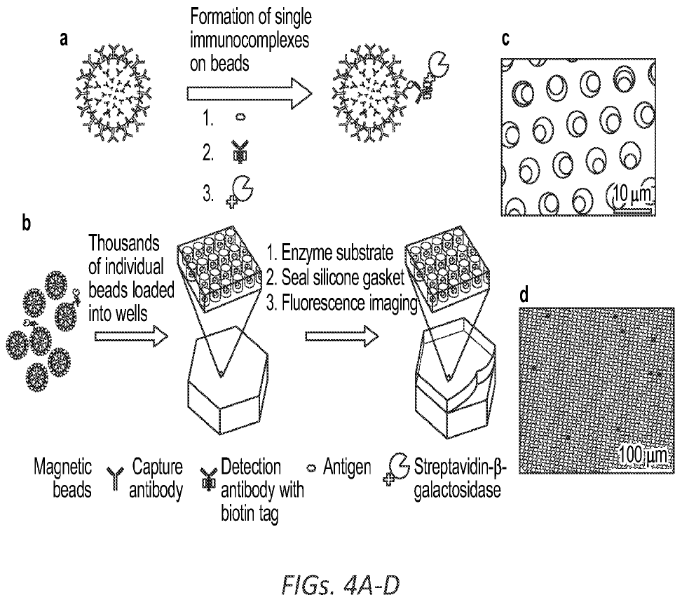

In some embodiments, the protein biomarkers in blood for breast cancer detection are measured using SIMOA assays (25, 26). SIMOA assays have several advantages over the conventional ELISA, the current gold standard for protein detection in blood. First, SIMOA is 1000× more sensitive than ELISA and allows for quantification of analytes present at low concentrations (25). SIMOA can detect protein concentrations as low as 10⁻¹⁹ M compared to conventional ELISA's ability to detect only 10⁻¹² M. Second, due to the high sensitivity of SIMOA, the serum samples can be more dilute, which reduces non-specific binding that arises from matrix effects (53, 54). Third, SIMOA has a wide dynamic range that spans four orders of magnitude in concentration, and thus a single assay can be used to detect both low and high abundance markers (55). In some embodiments, the SIMOA technique achieves this high sensitivity by digitally counting the number of molecules in a sample by labeling and physically isolating each immunocomplex into femtoliter-sized wells (FIGS. 4A-D). These advantages provide for detection and quantification of blood biomarkers for developing a robust biomarker panel.

In some embodiments, mass spectrometry, and particularly matrix-assisted laser desorption/ionization mass spectrometry (MALDI-MS) and surface-enhanced laser desorption/ionization mass spectrometry (SELDI-MS), are used for the detection of biomarkers. (See U.S. Pat. Nos. 5,118,937; 5,045,694; 5,719,060; 6,225,047). In some embodiments, other methods can be used, e.g., standard electrophoretic and quantitative immunoassay methods for proteins, including but not limited to, Western blot; enzyme linked immunosorbent assay (ELISA); Enzyme-Linked Immunospot (ELISPOT); biotin/avidin type assays; protein array detection, e.g., protein microarrays; radio-immunoassay; immunohistochemistry (IHC); immune-precipitation assay; flow cytometry/FACS (fluorescent activated cell sorting); Proximity Ligation Assay (PLA); lateral flow assay; surface plasmon resonance (SPR); optical imaging; and mass spectrometry (Kim (2010) Am J Clin Pathol 134: 157-162; Yasun (2012) Anal Chem 84(14): 6008-6015; Brody (2010) Expert Rev Mol Diagn 10(8): 1013-1022; Philips (2014) PLOS One 9(3): e90226; Pfaffe (2011) Clin Chem 57(5): 675-687; Cohen and Walt, Chem. Rev. 2019, 119, 293-321). The methods typically include revealing labels such as fluorescent, chemiluminescent, radioactive, and enzymatic or dye molecules that provide a signal either directly or indirectly. As used herein, the term “label” refers to the coupling (i.e. physical linkage) of a detectable substance, such as a radioactive agent or fluorophore (e.g. phycoerythrin (PE) or indocyanine (Cy5)), to an antibody or probe, as well as indirect labeling of the probe or antibody (e.g. horseradish peroxidase, HRP) by reactivity with a detectable substance.

In some embodiments, the presence and/or level of the biomarker(s) is comparable to the presence and/or level of the protein(s) in the disease reference, and the subject has one or more symptoms associated with breast cancer, then the subject has breast cancer. In some embodiments, the subject has no overt signs or symptoms of breast cancer, but the presence and/or level of one or more of the proteins evaluated is comparable to the presence and/or level of the protein(s) in the disease reference, then the subject has breast cancer or an increased risk of developing breast cancer. In some embodiments, once it has been determined that a person has breast cancer, or has an increased risk of developing breast cancer, then a treatment, e.g., as known in the art or as described herein, can be administered.

Suitable reference values can be determined using methods known in the art, e.g., using standard clinical trial methodology and statistical analysis. The reference values can have any relevant form. In some cases, the reference comprises a predetermined value for a meaningful level of the biomarker(s), e.g., a control reference level that represents a normal level of the biomarker(s), e.g., a level in an unaffected subject or a subject who is not at risk of developing a disease described herein, and/or a disease reference that represents a level of the proteins associated with breast cancer, e.g., a level in a subject having breast cancer.

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects. For example, in some embodiments, a control reference subject does not have breast cancer, does not have a risk of developing breast cancer, or does not later develop breast cancer.

A disease reference subject is one who has (or has an increased risk of developing) breast cancer. An increased risk is defined as a risk above the risk of subjects in the general population.

Thus, in some cases, where the biomarker is decreased in cancer (see Table 1), the level of the biomarker(s) in a subject being less than or equal to a reference level of the biomarker(s) is indicative of the presence or risk of developing breast cancer, and the level of the biomarker(s) in a subject being greater than or equal to the reference level of the biomarker(s) is indicative of the absence of disease or normal risk of the disease.

In other cases, where the biomarker is increased in cancer (see Table 1), the level of the biomarker(s) in a subject being greater than or equal to the reference level of the biomarker(s) is indicative of the presence or risk of developing breast cancer, and the level of the biomarker(s) in a subject being less than or equal to a reference level of the biomarker(s) is indicative of the absence of disease or normal risk of the disease.

As noted below, to build the diagnostic model, the outcome was binary breast cancer case status (breast cancer versus healthy). Age and protein markers were modeled as continuous predictors. The values were log transformed and a logistic regression model was used to classify breast cancer and healthy subjects. To assess the classification accuracy of each particular model, subjects with a predicted probability of at least 50% were assigned as predicted to have cancer, while those below 50% were predicted to be healthy. A subject's predicted case status for a given model was then compared to the observed case status.

Thus, in some embodiments, to assess whether a subject has breast cancer in the clinic, the method can include first log transforming the biomarker values and then assigning a predicted probability, e.g., using a logistic regression model, to produce a probability score. If a subject has a predicted probability score above a selected threshold, e.g., at least 50%, the subject would be predicted to have cancer (e.g., assigned to a cancer category). If the predicted probability score is below the selected threshold, e.g., 50%, the subject would be predicted to be healthy (e.g., assigned to a healthy category).

In some embodiments, the levels of the biomarkers are used to calculate a score, e.g., along with one or more additional variable, e.g., age. The score can be calculated, e.g., using an algorithm such as summation, or weighted summation, of the (normalized) levels of the biomarkers. Specific algorithms can be identified using known statistical methods including PCA, linear regression, SVM (support vector machine), decision tree, KNN (K-nearest neighbors), K-means, gradient boosting, or random forest methods.

For example, in some embodiments, an exemplary model uses a logistic regression analysis wherein each variable (biomarker, X) gets a weight (B). In the exemplary equation below, the weights (B) are calculated for each marker, and there can be unique B values for each of the biomarkers, e.g., for each of the 24 biomarkers and age (25 in total).

$$Ln ( \frac{P}{1 - P} ) = {β}_{0} + {β}_{1} {X}_{1} + {β}_{2} {X}_{2} + … + {β}_{k} {X}_{k}$$

In the clinic, the measured biomarker values (X values) can be used to obtain a probability score a patient will have cancer by plugging in the measured biomarker values (X) into the equation and then calculating a probability value (P). In some embodiments, the clinical procedure to obtain the individual's probability of having breast cancer would be as follows:

First, blood would be drawn from the screenee. Second, the screenee's blood concentration of each biomarker protein in the panel would be measured using Simoa. Third, the screenee's predicted probability of having breast cancer would be calculated based on a logistic regression formula with a dependent variable of the natural log of [(probability of having breast cancer)/(probability of not having breast cancer)], and with independent variables of age and each biomarker in the panel. The predicted probability could then inform discussions between the screenee and physician as to how best to proceed, such as a decision that no further follow-up is necessary or to pursue confirmatory radiologic imaging.

For the 24-marker panel, the model parameter estimates based on the Tufts sample with 197 participants were as follows, with age measured in years, CA15-3 and CA19-9 measured in units/mL, and all other markers measured in pg/mL:

| Parameter | Estimate |
|---|---|
| Intercept | 41.991788 |
| Age | −1.117431 |
| ADAM8 | −0.554368 |
| CA15-3 | 0.644346 |
| CA19-9 | 0.620155 |
| CA125 | 1.050753 |
| CD25 | 1.190345 |
| CEACAM1 | 0.999778 |
| CXCL10 | 0.315299 |
| CYR61 | −1.147209 |
| EGF | 1.576728 |
| EGFR | −1.282062 |
| ER | 0.139425 |
| GDF15 | −1.175137 |
| HE4 | 0.346111 |
| HER2 | 0.618756 |
| HER3 | −3.941255 |
| HER4 | −0.001800 |
| HSP70 | 2.303286 |
| IL-6 | 0.753264 |
| LCN2 | −2.402002 |
| MICA | −0.661617 |
| P21 | −0.073071 |
| PR | −0.246487 |
| PTX3 | −0.883281 |
| VEGF | −0.355392 |

For the 4-marker panel identified via cross validation, the model parameter estimates based on the Tufts sample with 197 participants were as follows, with age measured in years and all markers measured in pg/mL:

| Parameter | Estimate |
|---|---|
| Intercept | 23.377887 |
| Age | −0.503012 |
| HER3 | −2.487126 |
| HSP70 | 1.930531 |
| CYR61 | −0.233183 |
| LCN2 | −1.565567 |

In some embodiments, the amount by which the level (or score) in the subject is less than the reference level (or score) is sufficient to distinguish a subject from a control subject, and optionally is a statistically significantly less than the level (or score) in a control subject. In cases where the level (or score) of the biomarker(s) in a subject being equal to the reference level (or score) of the biomarker(s), the “being equal” refers to being approximately equal (e.g., not statistically different).

The predetermined value can depend upon the particular population of subjects (e.g., human subjects) selected. For example, an apparently healthy population will have a different ‘normal’ range of levels of the biomarker(s) than will a population of subjects which have, are likely to have, or are at greater risk to have, a disorder described herein. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

Breast cancer is typically categorized into one of three major subtypes, based on the presence or absence of molecular markers for estrogen or progesterone receptors and human epidermal growth factor 2 (ERBB2; formerly HER2): hormone receptor positive/ERBB2 negative, ERBB2 positive, and triple-negative (tumors lacking all 3 standard molecular markers); see, e.g., Waks and Winer, JAMA. 2019 Jan. 22; 321(3): 288-300. In addition, the present methods can be used to make a differential diagnosis between estrogen receptor positive (ER+) and triple negative breast cancer (TNBC). In these methods, at least MICA, CA125, and CD25, or at least ER, HER3, HER4, CXCL10, CYR61, P21, MICA, CD25, IL-6, and CA125, are determined and used to identify whether a subject has ER+breast cancer or TNBC. Exemplary coefficients for the 10- and 3-marker panels are as follows:

### Three-Marker Subtype Panel

| Parameter | Estimate |
|---|---|
| Intercept | 25.643 |
| MICA | 7.480 |
| CD25 | −7.261 |
| CA125 | −7.971 |

### Ten-Marker Subtype Panel

| Parameter | Estimate |
|---|---|
| Intercept | 26.8755 |
| ER | −0.1528 |
| HER3 | −2.7045 |
| HER4 | 2.0912 |
| CXCL10 | 0.7439 |
| CYR61 | 1.1506 |
| P21 | 0.5082 |
| MICA | 7.5670 |
| CD25 | −8.4828 |
| IL6 | −0.5281 |
| CA125 | −8.2135 |

Thus, in some embodiments, the model is used to identify presence of ER+ subtype. The model provides the log-odds of having an ER+ breast tumor versus not having breast cancer at all, and the predicted probability for an individual having ER+ breast cancer as compared to no breast cancer at all. For triple-negative subtype, the model provides the log-odds of having a triple-negative breast tumor versus not having breast cancer at all, and the predicted probability for an individual having triple-negative breast cancer as compared to no breast cancer at all.

The present methods can also be used to identify subjects for further evaluation, e.g., for imaging (e.g., mammogram or ultrasound) and/or biopsy, to confirm a cancer diagnosis.

### Methods of Treatment

The methods described herein include methods for the treatment of breast cancer. Generally, the methods include selecting and optionally administering a therapeutically effective amount of a treatment for breast cancer to a subject who has been determined to be in need of such treatment by a method described herein. Treatments for breast cancer include radiation, surgical resection, chemotherapy, hormone/endocrine therapy, and immunotherapy.

In some embodiments, where a subject is identified as likely to have TNBC, a treatment comprising administration of chemotherapy, e.g., platinum compounds, anthracycline-based or anthracycline and taxane-based chemotherapy, and/or regimens that include antimetabolites (for example, cyclophosphamide, methotrexate and 5-fluorouracil (CMF), or cyclophosphamide, epirubicin and 5-fluorouracil (CEF)) is selected and optionally administered (see, e.g. Bianchini et al., Nat Rev Clin Oncol. 2016 November; 13(11): 674-690; Bergin and Loi, F1000Res. 2019 Aug. 2; 8: F1000 Faculty Rev-1342; Kumar and Aggarwal, Arch Gynecol Obstet. 2016 February; 293(2): 247-69; Nedeljkovie and Damjanovie, Cells. 2019 Aug. 22; 8(9): 957; Al-Mahmood et al., Drug Deliv Transl Res. 2018 October; 8(5): 1483-1507; Caparica et al., ESMO Open. 2019 May 13; 4(Suppl 2): e000504).

In some embodiments, where a subject is identified as likely to have ER+ breast cancer, a treatment comprising administration of endocrine therapy (e.g., tamoxifen, toremifene, fulvestrant, Aromatase inhibitors (AIs) (e.g., Letrozole (Femara), Anastrozole (Arimidex), or Exemestane (Aromasin)) or ovarian suppression, e.g., by oophorectomy or LHRH analogs) and optionally chemotherapy (e.g., as above or phosphoinositide 3-kinase (PI3K), mechanistic target of rapamycin (mTOR), or cyclin-dependent kinase (CDK) 4/6 inhibitors or Poly(ADP-ribose) polymerase (PARP) inhibitors)) is selected and optionally administered (see Waks and Winer, JAMA. 2019 Jan. 22; 321(3): 288-300).

### EXAMPLES

The invention is further described in the following examples, which do not limit the scope of the invention described in the claims.

### Materials and Methods

The following materials and methods were used in the Example set forth herein.

### Study Design

In this study, we sought to develop a blood-based protein biomarker panel for breast cancer detection using analytically robust Simoa assays. We identified 24 biomarker candidates and developed and analytically validated the Simoa assays. We used mRNA expression levels in tumor tissues for these biomarkers from TCGA to further confirm that our selected biomarkers are indicative of breast cancer when compared to other cancers. We then used a first, preliminary sample cohort (n=49) of healthy and breast cancer patients and measured the 24 protein biomarker candidates in serum using the Simoa assays we developed. We then sought to validate our results in a second larger cohort. We initiated a sample collection at Tufts Medical Center. All subjects in this cohort were female and over 40 years old. The breast cancer subjects have not previously received treatment for breast cancer and had tumors generally consistent with early stage disease. We measured the concentrations of the 24 biomarkers in serum using our Simoa assays. We developed a model using a logistic regression analysis with these 24 biomarkers plus age in order to distinguish between the healthy and breast cancer subjects. To downselect the most important markers, we used a backwards selection process and then developed a model using the four most informative markers plus age. As a secondary analysis, we assessed the subtypes correctly classified as cancer by the two models. We also used the TCGA data to identify important biomarkers for distinguishing between the subtypes using the protein biomarkers in blood. We then built a model using a logistic regression analysis in order to determine whether a subject has ER+ or TNBC in serum using protein biomarkers. Informed consent was obtained for all blood samples used in this study.

### Biomarker Panel Analysis in Tumor Tissues

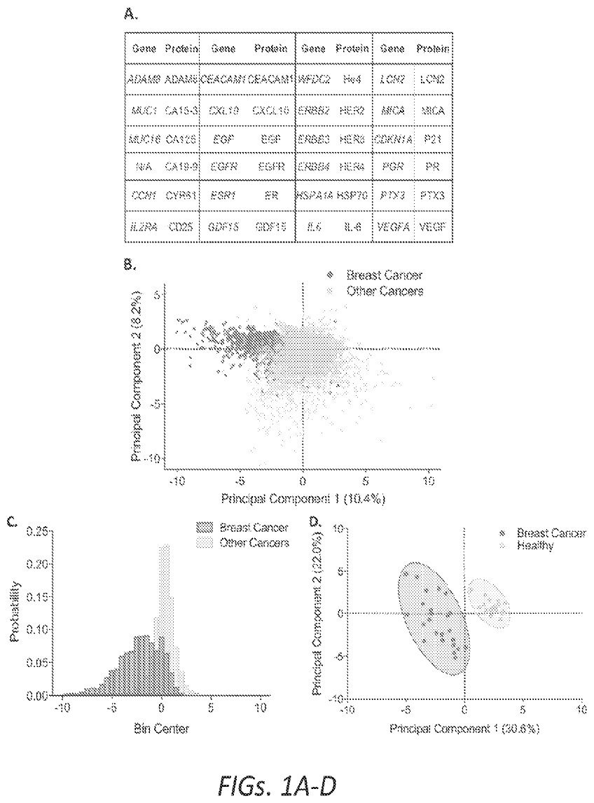

mRNA expression data deposited in The Cancer Genome Atlas (TCGA) database (cancergenome.nih.gov/) were obtained and a principal component analysis (PCA) was performed using the Caret package in R version 3.6.2. A total of 9,860 cancer subjects, of which 1,084 are breast cancer subjects, were analyzed. For this analysis, we used 23 out of the 24 biomarkers shown in FIG. 1A. We did not include CA19-9 in the analysis due to lack of corresponding mRNA data.

### Simoa Assay Description

Simoa assays are bead-based immunoassays with the major advance of signal detection by single molecule counting, which results in ultra-high sensitivity. Antibody-coated capture beads are added in large excess to a sample containing low concentrations of target analyte molecules. Poisson statistics dictate that either one or zero target protein molecules will bind to each bead. The beads are then incubated with a biotinylated detection antibody and streptavidin-β-galactosidase, forming an enzyme-labeled immunocomplex. Then the beads are loaded onto an array of 50 fL sized wells in which each well can hold only one bead. A fluorogenic substrate is added and the wells are sealed with oil, producing a locally high concentration of fluorescent product, thus enabling single molecule quantitation by counting active wells. At high target molecule concentrations, fluorescence intensity of the array is used to determine target concentration, thereby extending the dynamic range of the assay. The signal output is measured on the Simoa instrument using the standard unit of average enzymes per bead (AEB). All Simoa consumables and reagents were purchased from Quanterix Corp.

### Development of Ultra-Sensitive Simoa Assays

Capture antibodies were reconstituted and stored according to the instructions provided by the manufacturer. Antibody catalog numbers are provided in Table 1. The antibody was buffer exchanged to remove the storage buffer by first adding 0.13 mg of antibody solution to an Amicon filter (50K, EMD Millipore). Bead Conjugation Buffer (Quanterix) was then added to the filter up to a total volume of 500 μL. The filter device was centrifuged at 14,000× g for 5 minutes. The effluent was discarded and the process was repeated twice. The filter was inverted into a new tube and centrifuged at 1000× g for 2 minutes. The filter was rinsed with 50 μL of Bead Conjugation Buffer and centrifuged at 1000× g for 2 minutes. The concentration of the antibody was measured using a NanoDrop 2000 spectrophotometer. The antibody was diluted to 0.5 mg/mL in Bead Conjugation Buffer and stored on ice until ready for use. 2.8×10⁸ carboxylated, 2.7 μm, paramagnetic beads (Quanterix) were transferred into a microtube and washed three times with 200 μL of Bead Wash Buffer (Quanterix). The beads were then washed two times with 200 μL of Bead Conjugation Buffer and re-suspended in 190 μL of Bead Conjugation Buffer. Fresh 10 mg of 1-ethyl-3-(3-dimethylaminopropyl) carbodiimide hydrochloride (EDC) (ThermoFisher) was reconstituted in 1 mL of Bead Conjugation Buffer just prior to use. To activate the beads, 10 μL of EDC were added to the bead suspension to give a final concentration of 0.5 mg/ml and a final volume of 200 μL. The beads were then placed on a rotator for 30 minutes. The activated beads were then washed with 200 μL of Bead Conjugation Buffer. 200 μL of capture antibody solution was then added to the beads, vortexed, and placed on the rotator for 120 minutes for conjugation. The antibody-conjugated beads were then washed two times with 200 μL of Bead Wash Buffer. The beads were then blocked with 200 μL of Bead Blocking Buffer (Quanterix) and placed on the rotator for 30 minutes. The beads were washed with 200 μL of Bead Wash Buffer, washed with 200 μL of Bead Diluent (Quanterix), and re-suspended in 200 μEL of Bead Diluent. The beads were counted using a Beckman Coulter multi-sizer and stored at 4° C.

Detection antibodies that were not already biotinylated by the vendor were biotinylated for use in Simoa assays as previously described. (56) Briefly, the antibodies were purified using an Amicon filter three times in Biotinylation Reaction Buffer (Quanterix). Antibody concentrations were determined using NanoDrop One Spectrophotometer. Antibodies were conjugated to biotin using EZ-Link NHS-PEG4 Biotin (Thermo Fisher Scientific) using 40× molar excess and incubated for 30 min. The biotinylated antibodies were then purified using an Amicon filter.

Serum samples along with calibration curves were measured using the Simoa HD-1 Analyzer. The calibration curves were fit using a 4PL fit with a 1/y² weighting factor. The calibration curves were used to determine concentrations of the unknown samples. This analysis was done automatically using the software provided by Quanterix with the Simoa HD-1 Analyzer. The limit of detection (LOD) was calculated as the mean of the background plus three times the standard deviation.

### Biomarker Measurements in the Preliminary Sample Cohort

Breast cancer serum samples (n=25) and self-reported healthy serum samples (n=24) were obtained from BioIVT. The 24 protein markers were measured in duplicate in the samples using the Simoa assays. The mean of the measurements was calculated and the values were log transformed. A principal component analysis was then performed using the Caret package in R version 3.6.2.

### Subject Selection for Developing the Biomarker Panel

Breast cancer patients at Tufts Medical Center were screened and diagnosed with breast cancer via the standard approach, namely, mammography followed by biopsy. Patients who had not undergone surgical and/or therapeutic intervention were eligible. Eligible patients consented to blood donation for the study upon a positive breast cancer diagnosis. Healthy subjects were obtained from the Partner's Biobank, which provides a curated cohort of healthy subjects that were collected at several different hospitals. All subjects were female and over the age of 40 years old. Cases are referred to as breast cancer subjects and non-cases are referred to as healthy subjects.

### Statistical Analysis

Blood biomarker levels for 197subjects (100 healthy, 97 cancer) were analyzed. The outcome was binary breast cancer case status (breast cancer versus healthy). Age and protein markers were modeled as continuous predictors. Each marker had up to three replicates per subject. An individual's final marker measurement was the mean of non-missing replicate measurements. When a subject had no observed replicates for a particular marker in a given analysis model, the individual was first assigned an imputed value for the marker using multiple imputation. When a subject had a biomarker level that was below the LOD of a given assay, the value was assigned as the LOD of that assay. The values were log transformed and a logistic regression model was used to classify breast cancer and healthy subjects.

Five-fold cross validation was used to identify a subset of “high performing” markers. To perform the cross validation and marker selection, each of the 197 subjects was randomly assigned to one of five groups. For each of five folds, one group was excluded (test set) and the analysis performed on a combination of the other four groups (training set). Using PROC ADAPTIVEREG in SAS, each fold started in the fold-specific training set from a model of age and all 24 markers and worked backwards to an intercept-only model, with age in the model. The set of predictors yielding the smallest cross validation error was selected as the fold-specific model. The generalized cross validation criterion (GCV) was the measure of the fold-specific model's predictive accuracy. The contribution of each variable to the fold-specific model was measured by its importance, defined as the square root of the GCV value of the fold-specific model from which all basis functions involving the variable have been removed, minus the square root of the GCV value of the selected model, then scaled to set the largest importance value to 100. Markers with an importance of at least 70 in at least three folds were selected as cross-validated markers.

We then compared four models that differed by the set of included predictors: first, age alone; second, age plus HSP70, which was chosen by being the single marker with the greatest AUC; third, age plus four cross validation-selected markers (HSP70, HER3, CYR61, LCN2); and fourth, age plus all 24 markers. For each model, discrimination was assessed by AUC. Calibration was evaluated using LOESS-smoothed calibration plots of observed probability (0 or 1) versus estimated probability of the outcome. We explored the potential improvement in clinical decision-making for each model using decision curves, which plot net benefit versus threshold probability. A threshold probability is the probability designated as the cutoff to define high probability of an outcome, i.e. a positive test result.

To assess the classification accuracy of each particular model, subjects with a predicted probability of at least 50% were assigned as predicted to have cancer, while those below 50% were predicted to be healthy. A subject's predicted case status for a given model was then compared to the observed case status.

To assess our ability to distinguish between the different molecular breast cancer subtypes, we first performed a principal component analysis using mRNA expression levels for 23 biomarkers from the TCGA database. In the TCGA database, tumors are classified as Luminal A (n=412), Luminal B (n=174), Normal (n=25), Basal (n=136), HER2 (n=65). We then assessed the biomarker contribution to the first two principal components using the factoextra package in R and identified the ten most informative markers (top markers) and the ten least informative markers (bottom markers). Using the biomarker measurements in the breast cancer serum samples, we performed a logistic regression analysis using the two panels (top markers and bottom markers) in ER+(n=81) and TNBC (n=10) breast cancer subjects. ER+tumors were defined as having at least 1% of positive staining using immunohistochemistry of tissue biopsies. Triple negative tumors had no expression of ER, PR, or HER2. We then selected the three most informative markers from the model using the top markers and performed another logistic regression analysis using the three marker panel. The three markers were identified in R using the varImp function in the caret package.

Statistical analyses were run using SAS 9.4 (SAS Institute, Cary, NC) and R version 3.6.2. Figures were generated using GraphPad Prism 7 (San Diego, CA). Decision curves, and standard errors to estimate AUC confidence limits, were obtained using R and SAS macros available online (57, 58).

### Results

### Initial Validation of Candidate Biomarkers for Breast Cancer Detection

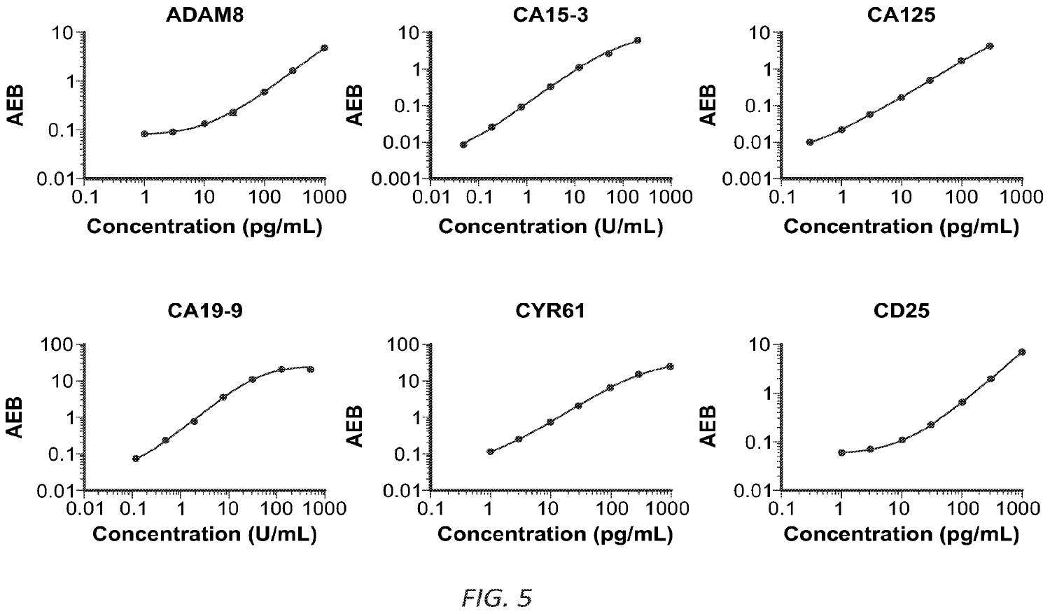

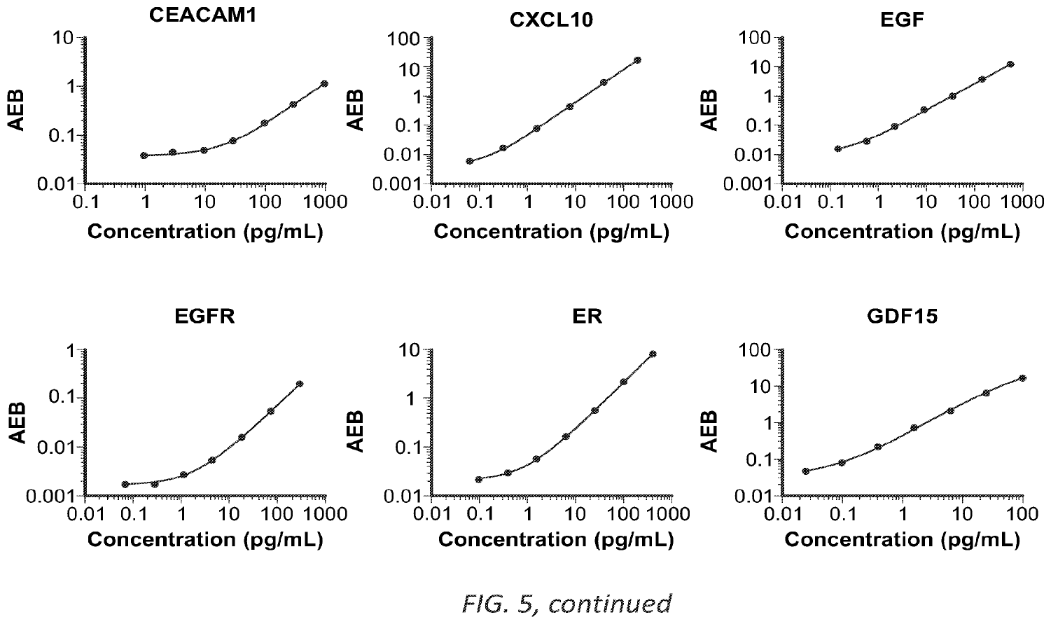

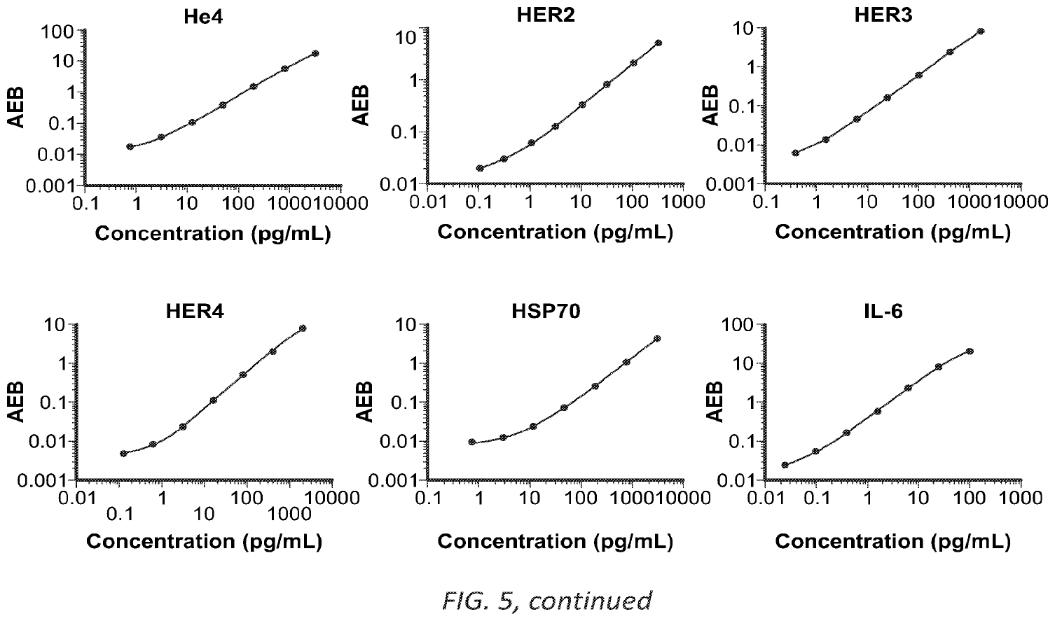

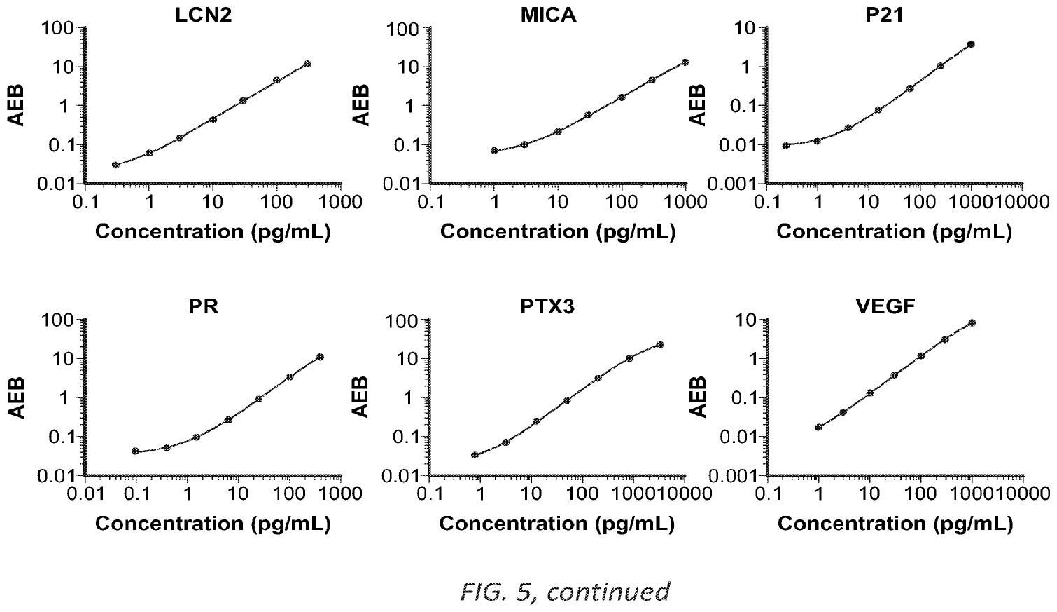

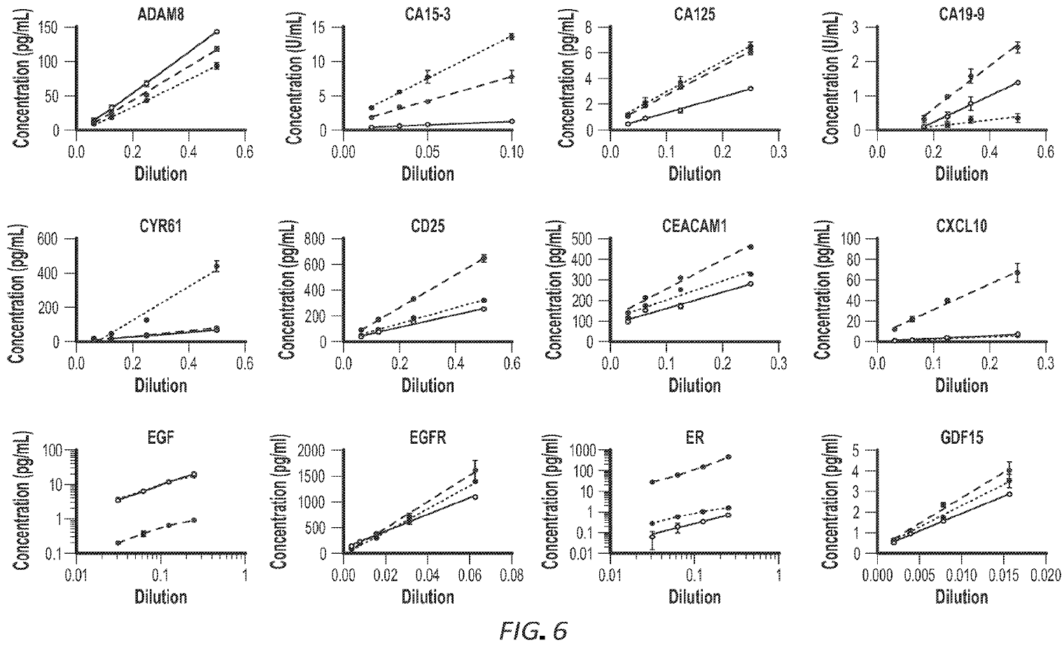

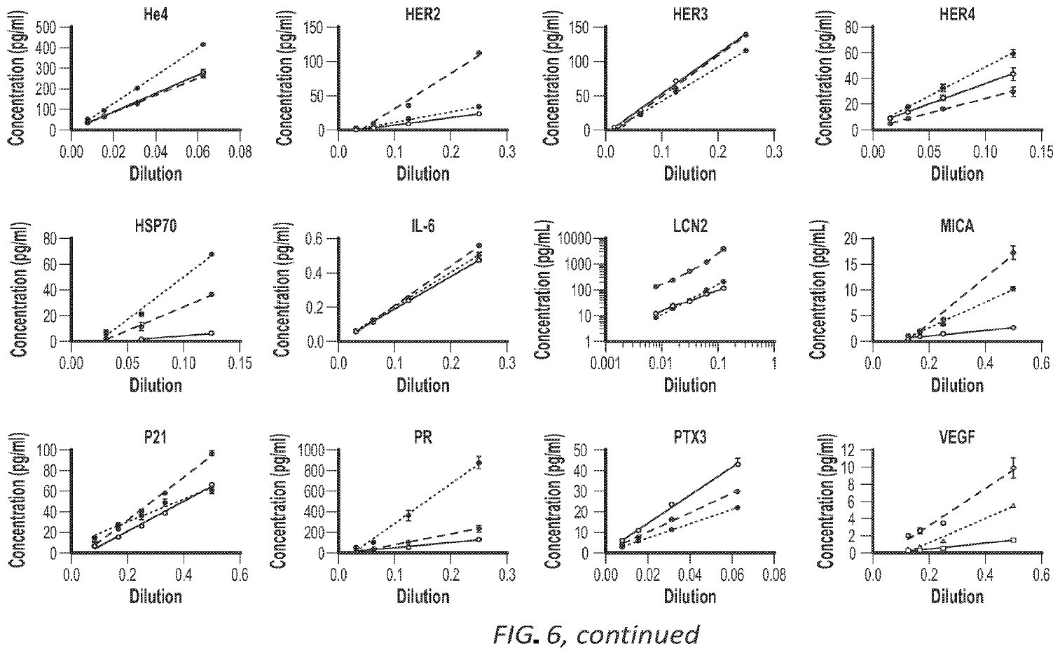

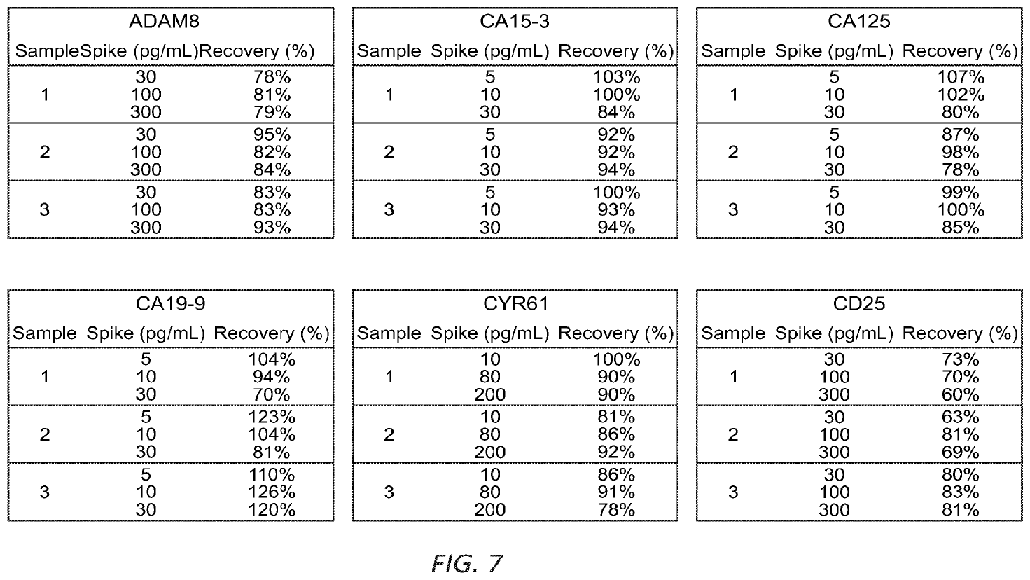

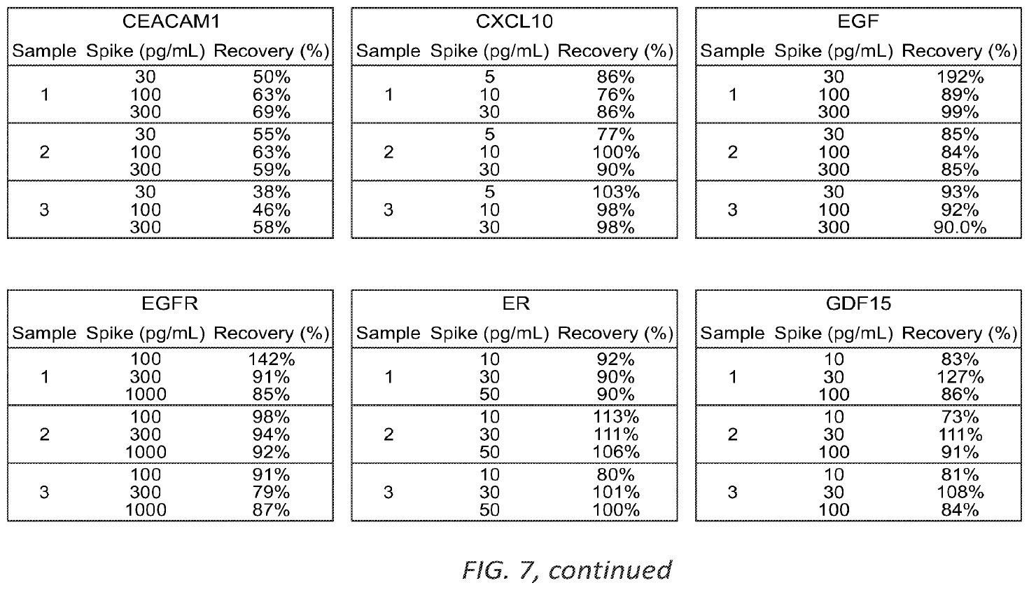

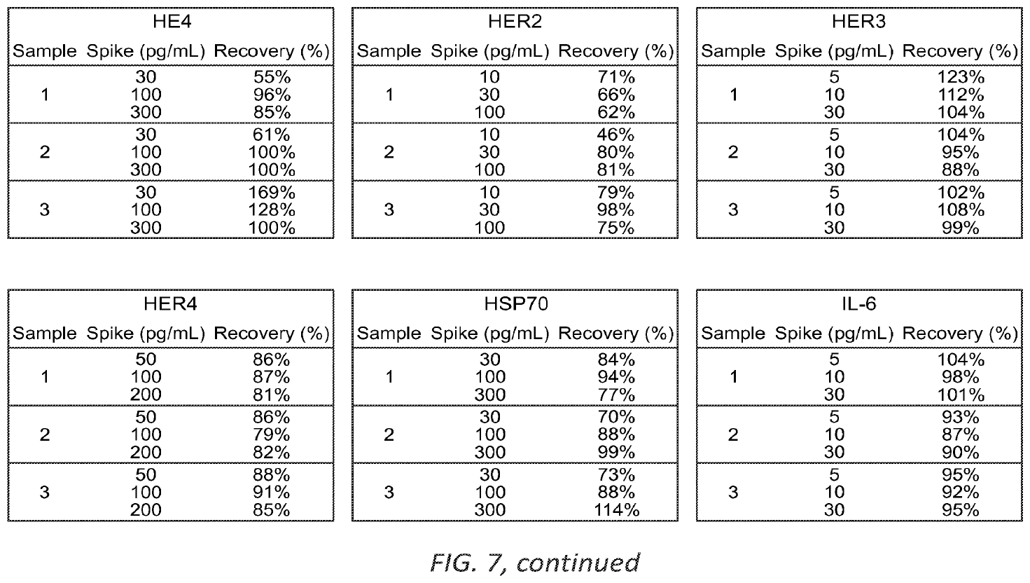

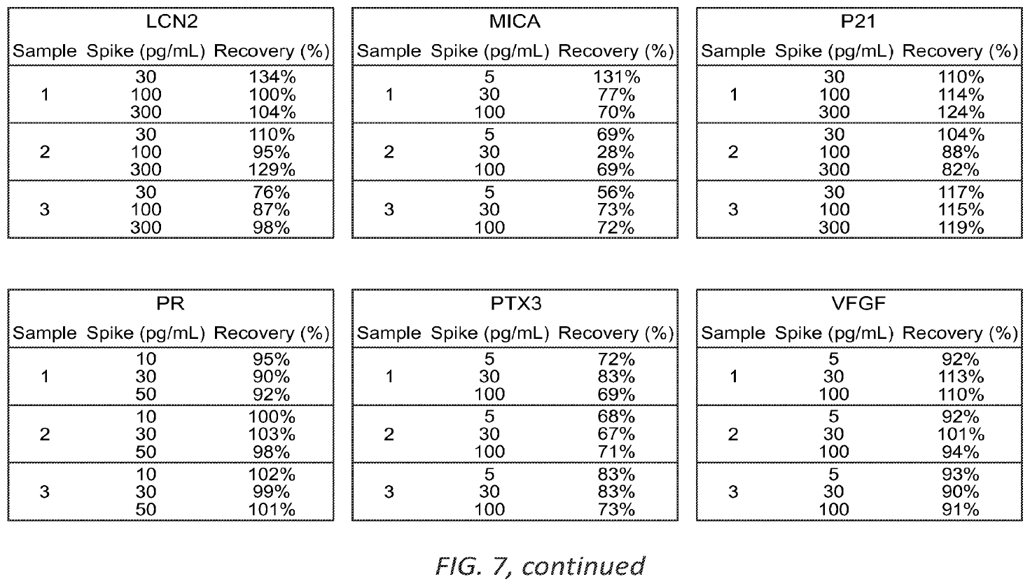

We selected 24 biomarker candidates (FIG. 1A) for breast cancer detection based on previous studies (28-49). We first assessed whether the biomarkers are associated with breast cancer based on gene expression levels in primary tumor tissues. Principal component analysis (PCA) of mRNA expression data deposited in The Cancer Genome Atlas (TCGA) database showed that the biomarkers were able to distinguish breast cancers from all other cancers (FIG. 1B-C). We then developed digital ELISA using Single Molecule Arrays (Simoa) assays for these biomarkers and ensured that the assays are analytically robust by performing rigorous validation tests (FIGS. 5-7, Tables S1-S2). Using these Simoa assays, we tested serum samples from a preliminary cohort of female self-reported healthy subjects (n=24) and breast cancer subjects (n=25) (FIG. 1D). We showed that this panel can easily distinguish between the healthy and breast cancer subjects. These results suggested that the panel of 24 biomarkers is promising for breast cancer detection. To confirm this result, we sought to investigate whether these biomarkers could be used to detect breast cancer in blood in a larger cohort of newly diagnosed patients who have not received any treatment.

### Blood Biomarker Panels for Breast Cancer Detection in a Newly Diagnosed, Treatment-Naïve Cohort

To assess our ability to detect breast cancer using a blood biomarker panel, we initiated a sample collection at Tufts Medical Center and analyzed serum samples from newly diagnosed patients. Patients were screened by mammography and diagnosed with breast cancer by biopsy. Patients who had a positive breast cancer diagnosis and had not undergone surgical or therapeutic interventions were eligible. Tumor characteristics for breast cancer subjects are given in Table 3. Tumors were generally consistent with early-stage disease, with most being small (T0-T2) and lymph node-negative (N0), and all being non-metastatic (M0). The majority of tumors were estrogen receptor (ER) positive, with a median ER measurement of 95% (interquartile range 85%, 98%) using immunohistochemistry of biopsy specimens. Healthy subjects were obtained from the Partners Biobank, which provides a curated cohort of blood samples from healthy subjects that were collected at several different hospitals. These 197 subjects (100 healthy and 97 breast cancer subjects) were all female and at least 40 years old.

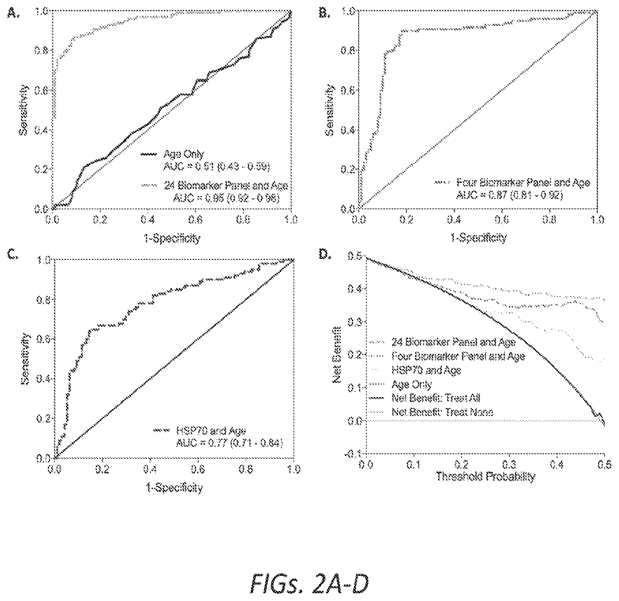

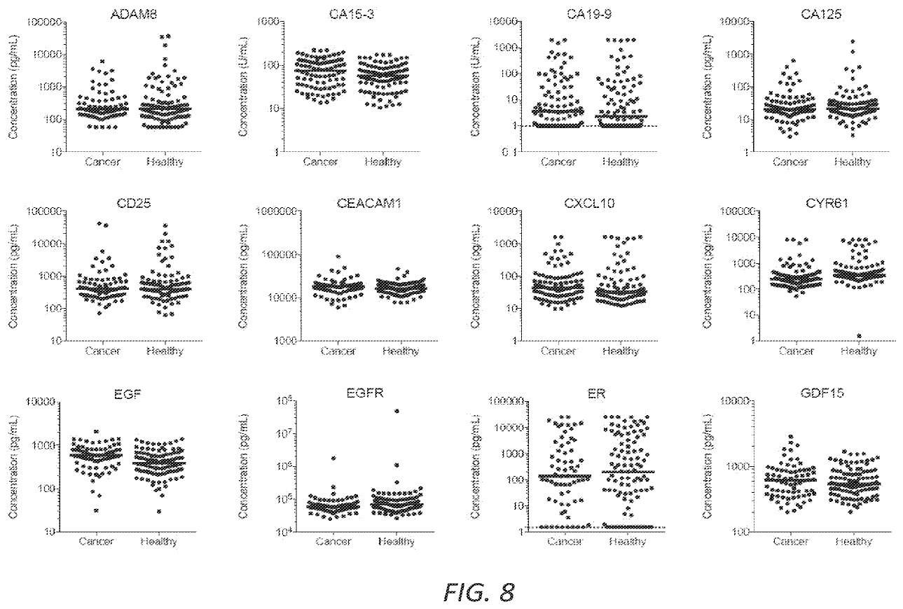

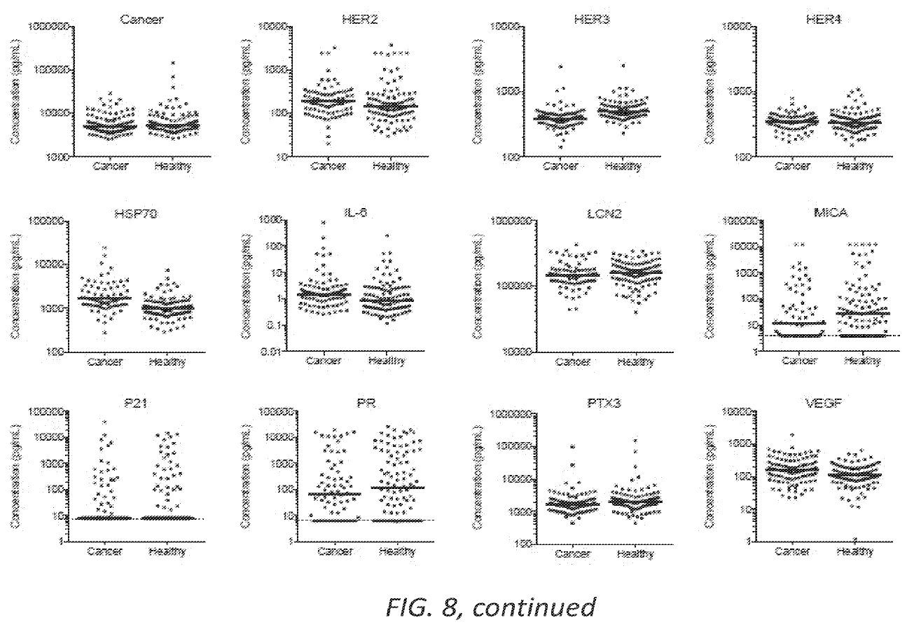

We measured serum biomarker levels in this sample cohort using the 24 Simoa assays. Table 1 and FIG. 8 present age and biomarker distributions for healthy and breast cancer subjects. Age distributions were similar for the healthy and breast cancer subjects. We then examined whether the biomarker panel could distinguish between healthy and breast cancer subjects using a logistic regression analysis. As shown in FIG. 2A, the model using all 24 biomarkers plus age had an area under the curve (AUC) of 0.95 (95% CI 0.92-0.98) while the model using age alone was uninformative with an AUC of 0.51 (95% CI 0.43-0.59). The model using all 24 biomarkers plus age correctly identified 174 of 197 (88%) subjects, with 87% sensitivity and 90% specificity.

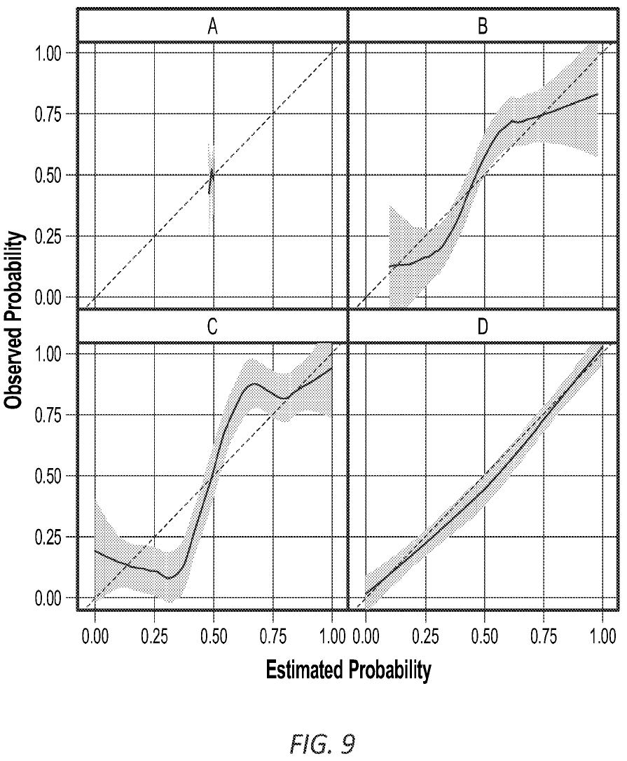

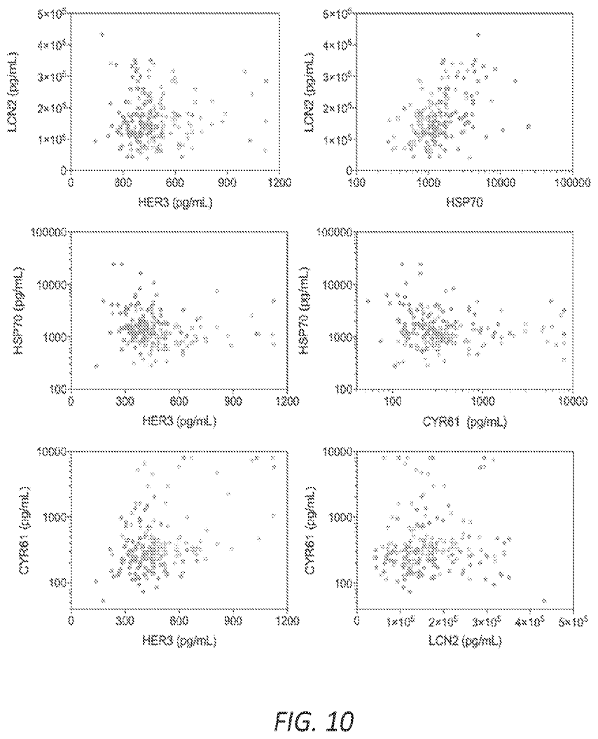

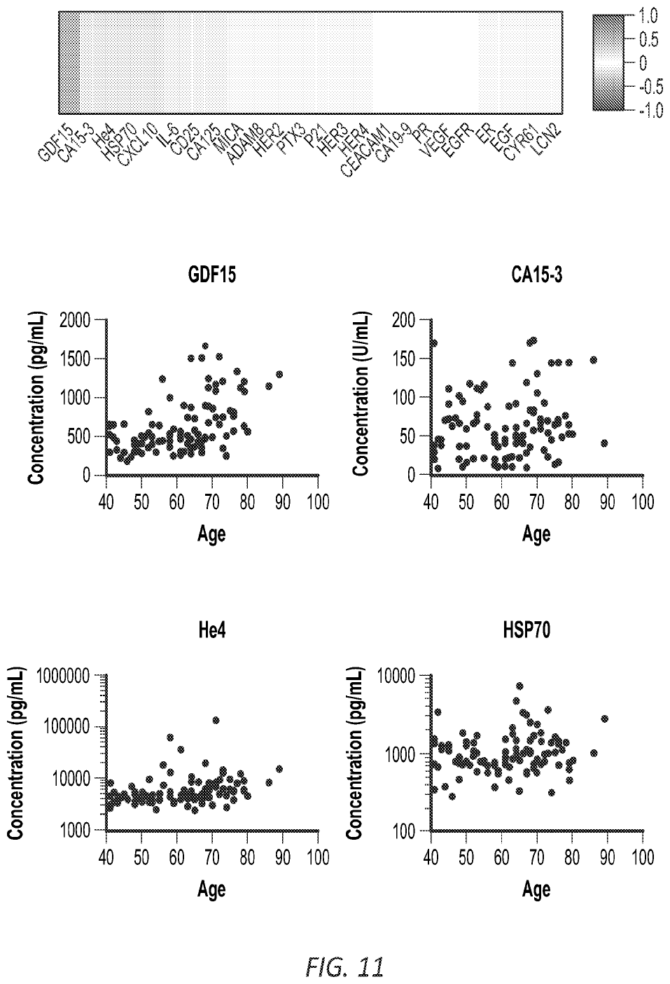

We then down-selected the most informative markers using a cross-validation backwards selection process with the 24 protein biomarkers plus age in the model, which yielded HER3, HSP70, CYR61, and LCN2 as the most informative markers (Table 4). The model using these four biomarkers plus age (FIG. 2B) had an AUC of 0.87 (95% CI 0.81-0.92). This model correctly identified 165 of 197 (84%) of the subjects, with 85% sensitivity and 83% specificity. The composite cross validation test-set AUC was 0.94 (95% CI 0.92-0.97), showing that the four biomarker panel was well-validated (Table 4). Model calibrations are shown in FIG. 9. We also assessed the performance of each of these biomarkers plus age on their own (Table 5). A model of HSP70 plus age (FIG. 2C) had an AUC of 0.77 (95% CI 0.71-0.84) and performed better than models of any other individual marker plus age. Compared to models of each individual marker plus age, the model using the four biomarkers plus age performed substantially better. These results suggest that the panel is critical to obtain optimal discrimination and that the individual markers alone are not sufficient to detect breast cancer. FIG. 10 shows XY scatterplots of the relationship between these four markers. Furthermore, we have included age in our model since the risk of breast cancer increases with age. We show that the concentrations of some biomarkers correlate with age in healthy subjects (FIG. 11).

We also assessed the net benefit ratio (FIG. 2D) (50-52). For all threshold probabilities of about 10% and above, the model using the 24 biomarkers plus age had a higher net benefit than any alternative model, including a decision to classify all subjects as healthy or, at the other extreme, to classify all subjects as cancer. For threshold probabilities below 10%, the differences in net benefit across the various models were small.

TABLE 1
Age and circulating protein concentrations. All proteins were measured
in pg/mL except for CA15-3 and CA19-9, which were measured in units/mL.
For a given protein, the final value per subject was the mean of
replicate measurements. IQR = Interquartile Range.

| | Breast Cancer | | Healthy Subjects | | |
|---|---|---|---|---|---|
| | Subjects (n = 97) | | (n = 100) | | |
| Characteristic | Median (IQR) | | Median (IQR) | | Change |
| Age, years | 61.0 | (53.0, 69.0) | 63.0 | (52.0, 70.0) | − |
| ADAM8 | 206 | (138, 388) | 214 | (122, 477) | − |
| CA15-3 | 74.4 | (35.6, 122) | 56.2 | (37.5, 85.8) | + |
| CA19-9 | 3.6 | (1.0, 37) | 2.3 | (1.0, 31) | + |
| CA125 | 20.0 | (15.0, 36.0) | 21.3 | (14.3, 32.3) | − |
| CD25 | 421 | (255, 757) | 398 | (279, 670) | + |
| CEACAM1 | 18,100 | (14,600, 20,700) | 16,700 | (13,900, 20,600) | + |
| CXCL10 | 43.7 | (25.1, 91.9) | 32.5 | (22.6, 80.2) | + |
| CYR61 | 243 | (159, 364) | 334 | (266, 631) | − |
| EGF | 575 | (388, 810) | 386 | (247, 630) | + |
| EGFR | 58,800 | (47,300, 70,900) | 69,600 | (51,600, 95,800) | − |
| ER | 138 | (15.4, 2,000) | 206 | (21.9, 4,400) | − |
| GDF15 | 606 | (375, 865) | 533 | (397, 837) | + |
| HE4 | 4,830 | (3,860, 7,370) | 5,250 | (4,340, 7,570) | − |
| HER2 | 187 | (113, 291) | 145 | (90.7, 269) | + |
| HER3 | 386 | (331, 454) | 490 | (435, 604) | − |
| HER4 | 344 | (278, 415) | 340 | (281, 417) | + |
| HSP70 | 1,640 | (1,170, 2,890) | 969 | (727, 1,410) | + |
| IL-6 | 1.5 | (0.8, 2.8) | 0.9 | (0.4, 2.5) | + |
| LCN2 | 145,000 | (116,000, 180,000) | 163,000 | (122,000, 214,000) | − |
| MICA | 12 | (4.0, 71) | 27 | (4.0, 130) | − |
| P21 | 7.8 | (7.8, 53) | 7.8 | (7.8 110) | n/c |
| PR | 64 | (6.2, 560) | 110 | (6.2, 2,600) | − |
| PTX3 | 1,620 | (1,280, 2,110) | 1,940 | (1,470, 2,710) | − |
| VEGF | 155 | (82.2, 285) | 110 | (66.6, 185) | + |

+, increased in cancer;
−, decreased in cancer;
n/c, no change

### Subtype Analysis Using the Biomarker Candidates

Breast cancer is a heterogeneous disease that consists of different molecular subtypes and thus we sought to evaluate whether our models could accurately classify different breast cancer subtypes as cancer. We identified three subtypes in our breast cancer cohort: ER+ tumors, ER-/HER2+ tumors, and triple negative breast cancers (TNBC). We examined the performance of the 24 biomarker panel and the four biomarker panel that we described in the previous section and found that both models were able to accurately classify the different breast cancer subtypes as cancer (FIG. 3A). These results suggest that the panels can be used to generally detect breast cancers of different subtypes. To further confirm these results, we developed new models using the two biomarker panels and two different groups. The first group consisted of healthy and ER+ breast cancer subjects and the second group consisted of healthy and TNBC subjects. We found that all four models had high AUCs (FIG. 3B), suggesting that these biomarker panels can accurately distinguish between healthy and breast cancer subtypes.

We next wanted to determine whether the 24 protein biomarkers could distinguish between ER+ and TNBC in blood. Due to our small sample size (for ER+, n=81 and for TNBC, n=10) we sought to downselect and identify the most important biomarkers that could distinguish between the different subtypes. To downselect the markers, we examined mRNA expression levels for the 24 biomarkers in primary tumors by TCGA and observed that the ER+ and TNBC subtypes clustered away from each other (FIG. 3C). We selected the top ten markers that contributed the most to the principal components (FIG. 3D) and developed a model using these ten protein biomarkers in blood, which provided an AUC of 0.96 (95% CI 0.92-1.00) (FIG. 3E).

We identified MICA, CA125, and CD25 as the top three most informative protein biomarkers in blood for subtyping (FIG. 12) and observe an AUC of 0.96 (95% CI 0.91-1.00) using this three-marker panel (FIG. 3E). Altogether, our results suggest that the protein biomarkers can accurately classify each of several different breast cancer subtypes, and further, that a subset of the 24 biomarkers can distinguish ER+ from TNBC in blood.

TABLE 1
Simoa assay set up.
Simoa assay setup

| | | | Incubation | Detector | | Sample | |
|---|---|---|---|---|---|---|---|
| | | Assay | times | antibody conc. | SβG conc. | dilution | |
| | Target | configuration | (cadences) | (ug/mL) | (pM) | factor | LOD |
| 1 | ADAM8 | 3 step | 20-7-7 | 0.3 | 50 | 16 | 3.600 |
| 2 | CA15-3 | 3 step | 20-7-7 | 0.7 | 150 | 30 | 0.004 |
| 3 | CA125 | 3-step | 20-7-7 | 0.2 | 144 | 8 | 0.095 |
| 4 | CA19-9 | 2 step | 47-7 | 0.5 | 50 | 4 | 0.250 |
| 5 | CYR61 | 2 step | 47-7 | 1 | 150 | 8 | 0.151 |
| 6 | CD25 | 2 step | 47-7 | 1 | 150 | 16 | 3.000 |
| 7 | CEACAM1 | 3-step | 20-7-7 | 0.3 | 50 | 64 | 1.812 |
| 8 | CXCL10 | 2 step | 47-7 | 1X (stock 200X) | 150 | 8 | 0.129 |
| 9 | EGF | 3 step | 20-7-7 | 0.05 | 9 | 30 | 1.000 |
| 10 | EGFR | 3-step | 20-7-7 | 0.1 | 50 | 64 | 3.960 |
| 11 | ER | 2 step | 47-7 | 1 | 100 | 16 | 0.097 |
| 12 | GDF15 | 2 step | 47-7 | 1 | 36 | 512 | 0.013 |
| 13 | He4 | 2 step | 47-7 | 0.7 | 50 | 32 | 0.333 |
| 14 | HER2 | 2 step | 47-7 | 0.8 | 25 | 8 | 0.053 |
| 15 | HER3 | 3 step | 20-7-7 | 0.3 | 150 | 16 | 0.300 |
| 16 | HER4 | 3 step | 20-7-7 | 0.3 | 150 | 30 | 0.275 |
| 17 | HSP70 | 2 step | 47-7 | 0.5 | 72 | 8 | 0.730 |
| 18 | IL-6 | 3 step | 20-7-7 | 0.3 | 150 | 4 | 0.009 |
| 19 | LCN2 | 2 step | 47-7 | 0.5 | 36 | 512 | 0.038 |
| 20 | MICA | 2 step | 47-7 | 1 | 150 | 4 | 1.000 |
| 21 | P21 | 2 step | 47-7 | 1 | 100 | 8 | 0.970 |
| 22 | PR | 2 step | 47-7 | 1 | 100 | 16 | 0.390 |
| 23 | PTX3 | 2 step | 47-7 | 0.7 | 50 | 32 | 0.349 |
| 24 | VEGF | 3 step | 20-7-7 | 0.3 | 75 | 4 | 0.119 |

TABLE 2
Simoa assay reagents. All reagents were obtained
from R&D Systems unless otherwise indicated.

| | | Capture | Detector | Protein |
|---|---|---|---|---|
| | Target | antibody | antibody | standard |
| 1 | ADAM8 | DY1031 | DY1031 | DY1031 |
| 2 | CA15-3 | 10-C03E | 10-C03F | 30-1066 |
| | | (Fitzgerald) | (Fitzgerald) | (Fitzgerald) |
| 3 | CA125 | DY5609 | DY5609 | DY5609 |
| 4 | CA19-9 | 10-CA19B | 10-CA19A | 30-AC14 |
| | | (Fitzgerald) | (Fitzgerald) | (Fitzgerald) |
| 5 | CYR61 | DY4055 | DY4055 | DY4055 |
| 6 | CD25 | DY223 | DY223 | DY223 |
| 7 | CEACAM1 | DY2244 | DY2244 | DY2244 |
| 8 | CXCL10 | DY266 | 439904 | DY266 |
| | | | (BioLegend) | |
| 9 | EGF | DY236 | DY236 | DY236 |
| 10 | EGFR | DYC1854 | DYC1854 | DYC1854 |
| 11 | ER | DYC5715 | DYC5715 | DYC5715 |
| 12 | GDF15 | DY957 | BAF940 | DY957 |
| 13 | He4 | DY6274 | DY6274 | DY6274 |
| 14 | HER2 | DYC1129 | DYC1129 | DYC1129 |
| 15 | HER3 | DYC234 | DYC234 | DYC234 |
| 16 | HER4 | DYC1133 | DYC1133 | DYC1133 |
| 17 | HSP70 | DYC1663 | DYC1663 | DYC1663 |
| 18 | IL-6 | MAB206 | BAF206 | 206IL |
| 19 | LCN2 | DY1757 | DY1757 | DY1757 |
| 20 | MICA | DY1300 | DY1300 | DY1300 |
| 21 | P21 | DYC1047 | DYC1047 | DYC1047 |
| 22 | PR | DYC5415 | DYC5415 | DYC5415 |
| 23 | PTX3 | DY1826 | DY1826 | DY1826 |
| 24 | VEGF | AHG0114 | BAF293 | DY293 |
| | | (ThermoFisher) | | |

### Tumor Characteristics of Breast Cancer Cases (n=97)

TABLE 3
Tumor Characteristics.

| Characteristic | Median (IQR) or N (%) | | N Missing |
|---|---|---|---|
| Cancer Type | | | 0 |
| Invasive | 84 | (87%) | |
| In Situ | 13 | (13%) | |
| Cancer Location | | | 2 |
| Ductal | 86 | (91%) | |
| Lobular | 9 | (9%) | |
| ER, % positive cells^{a} | 95 | (85, 98) | 1 |
| ER Positive Status (>=10%)^{b} | 77 | (84%) | 5 |
| PR, % positive cells^{a} | 77.5 | (0, 95) | 1 |
| PR Positive Status (>=10%)^{b} | 62 | (70%) | 9 |
| HER2 Positive Status | 26 | (30%) | 11 |
| Tumor Size | | | 2 |
| T0 | 11 | (12%) | |
| T1 | 61 | (64%) | |
| T2 | 17 | (18%) | |
| T3 | 4 | (4%) | |
| T4 | 2 | (2%) | |
| Lymph Node Metastasis | | | 13 |
| N0 | 55 | (65%) | |
| N1 | 26 | (31%) | |
| N2 | 3 | (4%) | |
| Tumor Grade | | | 1 |
| Well Differentiated | 24 | (25%) | |
| Moderately Differentiated | 52 | (54%) | |
| Poorly Differentiated | 20 | (21%) | |

For categorical variables, category percentages are based on participants with non-missing data for the variable.
^{a}Includes those tumors with measurements in the range of 1-9%.
^{b}Excludes those tumors (4 ER, 8 PR) with measurements in the range of 1-9% due to ambiguous nature of tumors with these hormone receptor levels. Receptor-negative status defined as 0%.
ER = Estrogen Receptor,
IQR = Interquartile Range,
PR = Progesterone Receptor

TABLE 4
Predictive accuracy and variable importance of five-fold cross validation. Each
participant randomly assigned to one of five groups. In each fold, one group was held
out as the test set and the other groups combined served as the training set.
Five-fold cross validation (n = 197)

| | Fold 1 | | Fold 2 | | Fold 3 | | Fold 4 | | Fold 5 | |
|---|---|---|---|---|---|---|---|---|---|---|
| | Variable | VI | Variable | VI | Variable | VI | Variable | VI | Variable | VI |
| Training | HSP70 | 100 | HSP70 | 100 | HER3 | 100 | HSP70 | 100 | HSP70 | 100 |
| Set | HER3 | 95 | LCN2 | 88 | HSP70 | 96 | HER3 | 89 | LCN2 | 95 |
| | LCN2 | 85 | HER2 | 81 | LCN2 | 84 | CYR61 | 88 | HER3 | 95 |
| | CYR61 | 79 | CA15-3 | 80 | CA19-9 | 74 | LCN2 | 80 | CYR61 | 81 |
| | CA15-3 | 75 | CA19-9 | 70 | CYR61 | 71 | CXCL10 | 78 | EGF | 79 |
| | EGF | 67 | EGFR | 63 | EGF | 61 | CEACAM1 | 72 | CEACAM1 | 74 |
| | CA125 | 41 | CXCL10 | 58 | CEACAM1 | 7 | VEGF | 71 | HE4 | 69 |
| | VEGF | 0 | CA125 | 58 | ADAM8 | 2 | HE4 | 66 | ER | 34 |
| | CEACAM1 | 0 | ER | 0 | PR | 1 | GDF15 | 59 | ADAM8 | 0 |
| | PR | 0 | Age | 0 | VEGF | 0 | IL-6 | 50 | IL-6 | 0 |
| | Age | 0 | | | CA125 | 0 | EGF | 49 | CXCL10 | 0 |
| | MICA | 0 | | | Age | 0 | CA125 | 34 | Age | 0 |
| | | | | | | | Age | 0 | PR | 0 |

AUC, All Test Sets Combined: 0.94 (95% Cl 0.92-0.97)
AUC = Area Under Receiver Operating Characteristic Curve,
VI = Variable Importance

TABLE 5
AUC for models of age and one marker. Each AUC is for a model
of breast cancer (n = 97) and healthy subjects (n =
100) with predictors of age and one marker. Predictors were
log-transformed. AUC, area under receiver operating characteristic curve.

| Marker | AUC |
|---|---|
| ADAM8 | 0.509 |
| CA15-3 | 0.594 |
| CA19-9 | 0.529 |
| CA125 | 0.518 |
| CD25 | 0.526 |
| CEACAM1 | 0.546 |
| CXCL10 | 0.536 |
| CYR61 | 0.675 |
| EGF | 0.661 |
| EGFR | 0.622 |
| ER | 0.555 |
| GDF15 | 0.522 |
| HE4 | 0.563 |
| HER2 | 0.594 |
| HER3 | 0.772 |
| HER4 | 0.525 |
| HSP70 | 0.775 |
| IL-6 | 0.644 |
| LCN2 | 0.544 |
| MICA | 0.533 |
| p21 | 0.505 |
| PR | 0.536 |
| PTX3 | 0.583 |
| VEGF | 0.623 |

### REFERENCES

1. A. Jemal, et al., Global cancer statistics, C A. Cancer J. Clin. (2011), doi: 10.3322/caac.20107.

2. B. O. Anderson, R. Jakesz, Breast Cancer Issues in Developing Countries: An Overview of the Breast Health Global Initiative, World J. Surg. 32, 2578-2585 (2008).

3. K. J. Jorgensen, P. C. Gotzsche, Overdiagnosis in publicly organised mammography screening programmes: systematic review of incidence trends, Bmj 339, b2587 (2009).

4. R. D. Rosenberg, et al., Performance benchmarks for screening mammography, Radiology 241, 55-66 (2006).

5. J. G. Elmore, et al., Ten-Year Risk of False Positive Screening Mammograms and Clinical Breast Examinations, N. Engl. J. Med. 338, 1089-1096 (1998).

6. R. A. Hubbard, et al., Cumulative probability of false-positive recall or biopsy recommendation after 10 years of screening mammography: a cohort study, Ann. Intern. Med. 155, 481-492 (2011).

7. R. D. Rosenberg, et al., Effects of age, breast density, ethnicity, and estrogen replacement therapy on screening mammographic sensitivity and cancer stage at diagnosis: review of 183,134 screening mammograms in Albuquerque, New Mexico., Radiology 209, 511-518 (1998).

8. K. Kerlikowske, et al., Likelihood ratios for modern screening mammography: risk of breast cancer based on age and mammographic interpretation, Jama 276, 39-43 (1996).

9. P. L. Porter, et al., Breast tumor characteristics as predictors of mammographic detection: comparison of interval-and screen-detected cancers, J. Natl. Cancer Inst. 91, 2020-2028 (1999).

10. J. Holm, et al., Risk factors and tumor characteristics of interval cancers by mammographic density, J. Clin. Oncol. 33, 1030-1037 (2015).

11. B. Gao, et al., Mammographic and clinicopathological features of triple-negative breast cancer, Br. J. Radiol. 87, 20130496 (2014).

12. G. Siravegna, et al., Integrating liquid biopsies into the management of cancer, Nat. Rev. Clin. Oncol. 14, 531-548 (2017).

13. G. Rossi, M. Ignatiadis, Promises and Pitfalls of Using Liquid Biopsy for Precision Medicine, Cancer Res. 79, 2798 L-2804 (2019).

14. A. van de Stolpe, et al., Circulating tumor cell isolation and diagnostics: toward routine clinical use (2011).

15. A. Bardelli, K. Pantel, Liquid Biopsies, What We Do Not Know (Yet)Cancer Cell 31, 172-179 (2017).

16. A. M. Aravanis, et al., Next-Generation Sequencing of Circulating Tumor DNA for Early Cancer Detection, Cell 168, 571-574 (2017).

17. F. Diehl, et al., Circulating mutant DNA to assess tumor dynamics, Nat. Med. 14, 985-990 (2008).

18. C. Alix-Panabieres, K. Pantel, Challenges in circulating tumour cell research, Nat. Rev. Cancer 14, 623 (2014).

19. T. Reinert, et al., Analysis of circulating tumour DNA to monitor disease burden following colorectal cancer surgery, Gut 65, 625 LP - 634 (2016).

20. S. A. Williams, et al., Plasma protein patterns as comprehensive indicators of health, Nat. Med. 25, 1851-1857 (2019).

21. B. Lehallier, et al., Undulating changes in human plasma proteome profiles across the lifespan, Nat. Med. 25, 1843-1850 (2019).

22. J. D. Cohen, et al., Detection and localization of surgically resectable cancers with a multi-analyte blood test, Science , eaar3247 (2018).

23. M. C. Liu, et al., Sensitive and specific multi-cancer detection and localization using methylation signatures in cell-free DNA, Ann. Oncol. 31, 745-759 (2020).

24. A. P. Lourenco, et al., A Noninvasive Blood-based Combinatorial Proteomic Biomarker Assay to Detect Breast Cancer in Women Under the Age of 50 Years, Clin. Breast Cancer 17, 516-525.e6 (2017).

25. D. M. Rissin, et al., Single-molecule enzyme-linked immunosorbent assay detects serum proteins at subfemtomolar concentrations., Nat. Biotechnol. 28, 595-599 (2010).

26. L. Cohen, D. R. Walt, Single-molecule arrays for protein and nucleic acid analysis (2017).

27. D. C. Koboldt, et al., Comprehensive molecular portraits of human breast tumours, Nature 490, 61-70 (2012).

28. J. S. Ross, et al., Oncologist 8, 307-25 (2003).

29. C. J. Witton, et al., Expression of the HER1-4 family of receptor tyrosine kinases in breast cancerJ. Pathol. 200, 290-297 (2003).

30. A. M. Abukhdeir, et al., Tamoxifen-stimulated growth of breast cancer due to p21 loss, Proc. Natl. Acad. Sci. U.S.A. 105, 288 LP-293 (2008).

31. J. Yang, et al., Lipocalin 2 is a Novel Regulator of Angiogenesis in Breast Cancer, FASEB J. 27, 45-50 (2013).

32. J. Yang, et al., a Moses, Lipocalin 2 Promotes Breast Cancer Progression., Proc. Natl. Acad. Sci. U.S.A. 106, 3913-3918 (2009).

33. M. E. Murphy, The HSP70 family and cancer, Carcinogenesis 34, 1181-1188 (2013).

34. F. U. Hartl, et al., Molecular chaperones in protein folding and proteostasisNature 475, 324-332 (2011).

35. J. P. Joshi, et al., Growth differentiation factor 15 (GDF15)-mediated HER2 phosphorylation reduces trastuzumab sensitivity of HER2-overexpressing breast cancer cells, Biochem. Pharmacol. 82, 1090-1099 (2011). 36. B. F. Peake, et al., Growth differentiation factor 15 mediates epithelial mesenchymal transition and invasion of breast cancers through IGF-1R-FoxM1 signaling, Oncotarget 8, 94393-94406 (2017).

37. M.-T. Lin, et al., Cyr61 expression confers resistance to apoptosis in breast cancer MCF-7 cells by a mechanism of NF-kappaB-dependent XIAP up-regulation., J. Biol. Chem. 279, 24015-23 (2004).

38. D. Xie, et al., Breast cancer: Cyr61 is overexpressed, estrogen-inducible, and associated with more advanced disease, J. Biol. Chem. 276, 14187-14194 (2001).

39. R. G. Moore, D et al., A novel multiple marker bioassay utilizing HE4 and CA125 for the prediction of ovarian cancer in patients with a pelvic mass, Gynecol. Oncol. 112, 40-46 (2009).

40. S. T. Lee-Hoeflich, et al., A central role for HER3 in HER2-amplified breast cancer: Implications for targeted therapy, Cancer Res. 68, 5878-5887 (2008).

41. M. Kamei, et a1.,HE4 Expression Can Be Associated with Lymph Node Metastases and Disease-free Survival in Breast Cancer, Anticancer Res. 4784, 4779-4783 (2010).

42. J. Li, et al., HE4 (WFDC2) promotes tumor growth in endometrial cancer cell lines, Int. J. Mol. Sci. 14, 6026-6043 (2013).

43. K. R. Bauer, et al., Descriptive analysis of estrogen receptor (ER)-negative, progesterone receptor (PR)-negative, and HER2-negative invasive breast cancer, the so-called triple-negative phenotype: A population-based study from the California Cancer Registry, Cancer 109, 1721-1728 (2007).

44. M. Romagnoli et al., ADAM8 expression in invasive breast cancer promotes tumor dissemination and metastasis, EMBO Mol. Med. 6, 278-294 (2014).

45. C. Fang, et al., Serum CA125 is a predictive marker for breast cancer outcomes and correlates with molecular subtypes, Oncotarget 8, 63963-63970 (2017).

46. L. F. Norum, et al., Elevated CA125 in breast cancer—A sign of advanced disease, Tumour Biol 22, 223-228 (2001).

47. K. S. Goonetilleke, A. K. Siriwardena, Systematic review of carbohydrate antigen (CA 19-9) as a biochemical marker in the diagnosis of pancreatic cancer, Eur. J. Surg. Oncol. 33, 266-270 (2007).

48. M. J. Duffy, et al., CA 15-3: a prognostic marker in breast cancer, Int. J. Biol. Markers 15, 330-333 (2000).

49. J.-L. Wang, et al., Clinicopathological significance of CEACAM1 gene expression in breast cancer., Chin. J. Physiol. 54, 332-8 (2011).

50. A. J. Vickers, E. B. Elkin, Decision Curve Analysis: A Novel Method for Evaluating Prediction Models, Med. Decis. Mak. 26, 565-574 (2006).

51. A. J. Vickers, B. Van Calster, E. W. Steyerberg, Net benefit approaches to the evaluation of prediction models, molecular markers, and diagnostic tests, BMJ 352, i6 (2016).

52. A. J. Vickers, et al., A simple, step-by-step guide to interpreting decision curve analysis, Diagnostic Progn. Res. 3, 18 (2019).

53. D. Wild, The Immunoassay Handbook: Theory and applications of ligand binding, ELISA and related techniques (Elsevier, Amsterdam, The Netherlands, ed. 4th, 2013; linkinghub.elsevier.com/retrieve/pii/B9781455778966000583).

54. L. Cohen, et al., Single Molecule Arrays for ultra-sensitive detection of rat cytokines in serum, J. Immunol. Methods 452, 20-25 (2018).

55. D. M. Rissin, et al., Simultaneous detection of single molecules and singulated ensembles of molecules enables immunoassays with broad dynamic range, Anal. Chem. 83, 2279-2285 (2011).

56. L. Cohen, D. R. Walt, Evaluation of Antibody Biotinylation Approaches for Enhanced Sensitivity of Single Molecule Array (Simoa) Immunoassays, Bioconjug. Chem. 29, 3452-3458 (2018).

57. Cook N. Risk Prediction Modeling: cstat macro. ncook.bwh.harvard.edu/. Accessed 16 January 2020.

58. Decision Curve Analysis: DCA macro. decisioncurveanalysis.org/. Accessed 17 Feb. 2020.

59. Romagnoli, M. et al. ADAM8 expression in invasive breast cancer promotes tumor dissemination and metastasis. EMBO Mol. Med. 6, 278-294 (2014).

60. Joshi, J. P., Brown, N. E., Griner, S. E. & Nahta, R. Growth differentiation factor 15 (GDF15)-mediated HER2 phosphorylation reduces trastuzumab sensitivity of HER2-overexpressing breast cancer cells. Biochem. Pharmacol. 82, 1090-1099 (2011).

61. Peake, B. F., Eze, S. M., Yang, L., Castellino, R. C. & Nahta, R. Growth differentiation factor 15 mediates epithelial mesenchymal transition and invasion of breast cancers through IGF-1R-FoxM1 signaling. Oncotarget 8, 94393-94406 (2017).

62. Fang, C., Cao, Y., Liu, X., Zeng, X.-T. & Li, Y. Serum CA125 is a predictive marker for breast cancer outcomes and correlates with molecular subtypes. Oncotarget 8, 63963-63970 (2017).

63. Norum, L. F., Erikstein, B. & Nustad, K. Elevated CA125 in breast cancer--A sign of advanced disease. Tumour Biol 22, 223-228 (2001).

64. Moore, R. G. et al. A novel multiple marker bioassay utilizing HE4 and CA125 for the prediction of ovarian cancer in patients with a pelvic mass. Gynecol. Oncol. 112, 40-46 (2009).

65. Kamei, M., Yamashita, S., Tokuishi, K. & Hashioto, T. HE4 Expression Can Be Associated with Lymph Node Metastases and Disease-free Survival in Breast Cancer. Anticancer Res. 4784, 4779-4783 (2010).

66. Li, J. et al. HE4 (WFDC2) promotes tumor growth in endometrial cancer cell lines. Int. J. Mol. Sci. 14, 6026-6043 (2013).

67. Duffy, M. J., Shering, S., Sherry, F., McDermott, E. & O'Higgins, N. CA 15-3: a prognostic marker in breast cancer. Int. J. Biol. Markers 15, 330-333 (2000).

68. Ross, J. S. et al. The Her-2/neu gene and protein in breast cancer 2003: biomarker and target of therapy. Oncologist 8, 307-25 (2003).

69. Witton, C. J., Reeves, J. R., Going, J. J., Cooke, T. G. & Barlett, J. M. S. Expression of the HER1-4 family of receptor tyrosine kinases in breast cancer. Journal of Pathology 200, 290-297 (2003).

70. Lee-Hoeflich, S. T. et al. A central role for HER3 in HER2-amplified breast cancer: Implications for targeted therapy. Cancer Res. 68, 5878-5887 (2008).

71. Goonetilleke, K. S. & Siriwardena, A. K. Systematic review of carbohydrate antigen (CA 19-9) as a biochemical marker in the diagnosis of pancreatic cancer. Eur. J. Surg. Oncol. 33, 266-270 (2007).

72. Murphy, M. E. The HSP70 family and cancer. Carcinogenesis 34, 1181-1188 (2013).

73. Hartl, F. U., Bracher, A. & Hayer-Hartl, M. Molecular chaperones in protein folding and proteostasis. Nature 475, 324-332 (2011).

74. Bauernhofer, T. et al. Role of prolactin receptor and CD25 in protection of circulating T lymphocytes from apoptosis in patients with breast cancer. Br. J. Cancer 88, 1301-1309 (2003).

75. Knüpfer, H. & PreiB, R. Significance of interleukin-6 (IL-6) in breast cancer (review). Breast Cancer Res. Treat. 102, 129-135 (2006).

76. Wang, J.-L. et al. Clinicopathological significance of CEACAM1 gene expression in breast cancer. Chin. J. Physiol. 54, 332-8 (2011). 77. Yang, J., McNeish, B., Butterfield, C. & Moses, M. A. Lipocalin 2 is a Novel Regulator of Angiogenesis in Breast Cancer. FASEB J. 27, 45-50 (2013).

78. Yang, J. et al. Lipocalin 2 Promotes Breast Cancer Progression. Proc. Natl. Acad. Sci. U.S.A. 106, 3913-3918 (2009).

79. Liu, M., Guo, S. & Stiles, J. K. The emerging role of CXCL10 in ancer. Oncology Letters (2011). doi: 10.3892/01.2011.300

80. Madjd, Z. et al. Upregulation of MICA on high-grade invasive operable breast carcinoma. Cancer Immun. Arch. 7, 17 (2007).

81. Lin, M.-T. et al. Cyr61 expression confers resistance to apoptosis in breast cancer MCF-7 cells by a mechanism of NF-kappaB-dependent XIAP up-regulation. J. Biol. Chem. 279, 24015-23 (2004).

82. Xie, D. et al. Breast cancer: Cyr61 is overexpressed, estrogen-inducible, and associated with more advanced disease. J. Biol. Chem. 276, 14187-14194 (2001).

83. Abukhdeir, A. M. et al. Tamoxifen-stimulated growth of breast cancer due to p21 loss. Proc. Natl. Acad. Sci. U.S.A. 105, 288 LP-293 (2008).

84. Masuda, H. et al. Role of epidermal growth factor receptor in breast cancer. Breast Cancer Res. Treat. 136, 331-345 (2012).

85. Paplomata, E. & O'Regan, R. The PI3K/AKT/mTOR pathway in breast cancer: targets, trials and biomarkers. Ther. Adv. Med. Oncol. 6, 154-166 (2014).

86. Foley, J. et al. EGFR signaling in breast cancer: Bad to the bone. Semin. Cell Dev. Biol. 21, 951-960 (2010).

87. Pavlou, M. P., Dimitromanolakis, A. & Diamandis, E. P. Coupling proteomics and transcriptomics in the quest of subtype-specific proteins in breast cancer. Proteomics 13, 1083-1095 (2013).

88. Bauer, K. R., et al., Descriptive analysis of estrogen receptor (ER)-negative, progesterone receptor (PR)-negative, and HER2-negative invasive breast cancer, the so-called triple-negative phenotype: A population-based study from the California Cancer Registry. Cancer 109, 1721-1728 (2007).

89. Skobe, M. et al. Induction of tumor lymphangiogenesis by VEGF-C promotes breast cancer metastasis. Nat. Med. 7, 192-198 (2001).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method comprising:
obtaining a sample comprising blood from a subject, and determining a level of at least 2, 3, 4, 5, 10. 15, 20, or all 24 biomarkers as listed in Table A in the sample.

2. The method of claim 1, wherein the biomarkers comprise at least MICA, CA125, and CD25.

3. The method of claim 1, wherein the biomarkers comprise at least HER3, HSP70, CYR61, and LCN2.

4. The method of claim 1, wherein the biomarkers comprise at least ER, HER3, HER4, CXCL10, CYR61, P21, MICA, CD25, IL-6, and CA125.

5. The method of claim 1, further comprising calculating a score for the subject based on the level of the biomarkers.

6. The method of claim 2, further comprising calculating a score for the subject based on the level of the biomarkers, and comparing the score to subtype reference scores for known subtypes of breast cancer and identifying a subject who has a score that is comparable to the subtype reference as having that subtype of breast cancer.

7. The method of claim 5, further comprising recommending or sending the subject for additional evaluation.

8. The method of claim 7, wherein the additional evaluation comprises imaging and/or biopsy.

9. The method of claim 5, further comprising administering a treatment for breast cancer to a subject who has been identified as having or at risk of developing breast cancer.

10. The method of claim 8, wherein the treatment comprises chemotherapy, hormone therapy, immunotherapy, radiation, or surgical resection.

11. The method of claim 1, wherein determining a level of biomarkers comprises using digital ELISA; Meso Scale Discovery (MSD); Single-Molecule Counting (SMC); LUMINEX; SOMAscan Assays; mass spectrometry (optionally MALDI-MS), and/or mass cytometry (optionally CyTOF).

12. The method of claim 11, wherein the digital ELISA uses Single-Molecule Arrays (SIMOA).

13. A method of treating a subject, the method comprising:
obtaining a sample comprising blood from a subject,
determining a level of at least 2, 3, 4, 5, 10. 15, 20, or all 24 biomarkers as listed in Table A in the sample,
calculating a score for the subject based on the levels of the biomarkers,
identifying a subject who has a score above a threshold score; and
recommending or sending the subject for additional evaluation or administering a treatment for breast cancer to the subject who has a score above the threshold score.

14. The method of claim 13, wherein the biomarkers comprise at least MICA, CA125, and CD25, or comprise at least HER3, HSP70, CYR61, and LCN2.

15. The method of claim 13, wherein the biomarkers comprise at least ER, HER3, HER4, CXCL10, CYR61, P21, MICA, CD25, IL-6, and CA125.

16. The method of claim 13, further comprising calculating a score for the subject based on the level of the biomarkers, and comparing the score to subtype reference scores for known subtypes of breast cancer and identifying a subject who has a score that is comparable to the subtype reference as having that subtype of breast cancer.

17. The method of claim 13, comprising recommending or sending the subject who has been identified as having a score above the threshold score for additional evaluation, wherein the additional evaluation comprises imaging and/or biopsy.

18. The method of claim 13, comprising administering a treatment for breast cancer to a subject who has been identified as having a score above the threshold score, wherein the treatment comprises chemotherapy, hormone therapy, immunotherapy, radiation, or surgical resection.

19. The method of claim 13, wherein determining a level of biomarkers comprises using digital ELISA; Meso Scale Discovery (MSD); Single-Molecule Counting (SMC); LUMINEX; SOMAscan Assays; mass spectrometry (optionally MALDI-MS), and/or mass cytometry (optionally CyTOF).

20. The method of claim 19, wherein the digital ELISA uses Single-Molecule Arrays (SIMOA).
